(19) United States
(12) Patent Application Publication
(10) Pub. No.: US 2015/0259751 A1
(43) Pub. Date: Sep. 17, 2015

(54) MOLECULAR SIGNATURE FOR AGGRESSIVE SQUAMOUS CELL CARCINOMAS OF THE HEAD AND NECK

(71) Applicant: Washington University, St. Louis, MO (US)

(72) Inventor: Ravindra Uppaluri, St. Louis, MO (US)

(21) Appl. No.: 14/659,936

(22) Filed: Mar. 17, 2015

Related U.S. Application Data
(60) Provisional application No. 61/954,355, filed on Mar. 17, 2014.

(51) Int. Cl. C12Q 1/68; A61K 31/337; A61K 31/55; A61K 31/7068; A61K 33/24; A61K 31/282

(52) U.S. Cl. CPC C12Q 1/6886; A61K 33/24; A61K 31/282; A61K 31/55; A61K 31/7068; A61K 31/337; C12Q 2600/118; C12Q 2600/16; C12Q 2600/158

(57) ABSTRACT

The present invention encompasses methods of classifying HNSCC tumors, such as OSCC tumors, as aggressive or, alternatively, indolent.

### CROSS REFERENCE TO RELATED APPLICATIONS

This application claims the priority of U.S. provisional application No. 61/954,355, filed Mar. 17, 2015, which is hereby incorporated by reference in its entirety.

### FIELD OF THE INVENTION

The present invention encompasses methods of classifying squamous cell head and neck carcinomas, specifically OSCC tumors, as aggressive or, alternatively, indolent. The methods of the invention further comprise treating squamous cell head and neck carcinomas or OSCC tumors based on said classification.

### BACKGROUND OF THE INVENTION

Oral Squamous Cell Carcinoma (OSCC) is a major cause of cancer death worldwide, which is mainly due to disease recurrence leading to treatment failure and patient death.

OSCC accounts for 24% of all head and neck cancers. Currently available protocols for treatment of OSCCs include surgery, radiotherapy and chemotherapy. Complete surgical resection is the most important prognostic factor, since failure to completely remove a primary tumor is the main cause of patient death. Accuracy of the resection is based on the histological status of the margins, as determined by microscopic evaluation of frozen sections. Presence of epithelial dysplasia or tumor cells in the surgical resection margins is associated with a significant risk (66%) of local recurrence. However, even with histologically normal surgical margins, 10-30% of OSCC patients will still have local recurrence, which may lead to treatment failure and patient death.

Aggressive carcinogen-induced OSCC are difficult to treat due to locoregional recurrences. In contrast, more indolent lesions can be treated with single modality surgical intervention with low morbidity and favorable outcomes. Histologic criteria such as perineural or lymphovascular invasion and tumor depth, harbingers of early spread to regional lymph nodes, are commonly used to predict tumor behavior. Additionally, among clinical staging criteria, metastatic lymphadenopathy is one of the best predictors of a poor prognosis as it likely reflects aggressive primary tumor biology (seer.cancer.gov/statfacts/ html/oralcay.html). However, there is a dearth of studies delineating markers predictive of lymph node involvement, and genetic stratification approaches are at an early stage. In addition, the molecular underpinnings of aggressive OSCC growth and metastasis remain largely undefined. Thus, a molecular signature to identify OSCC aggressiveness is needed.

### SUMMARY OF THE INVENTION

In an aspect, the present invention encompasses a method for determining the aggressiveness of head and neck squamous cell carcinoma (HNSCC) in a subject. The method comprises providing a test sample from a subject known to have HNSCC; determining the nucleic acid expression levels in the test sample of at least a 10-nucleic acid molecular signature disclosed herein; and comparing the expression levels of each nucleic acid in the molecular signature to the corresponding reference expression levels of such nucleic acids, wherein differentially expressed levels in the test sample compared to the reference expression levels indicates aggressive HNSCC.

In another aspect, the present disclosure encompasses a method of treating a subject in need thereof. The method comprises obtaining a test sample from the subject; determining the aggressiveness of head and neck squamous cell carcinoma (HNSCC) in the subject using a molecular signature disclosed herein; and administering to the subject predicted to have aggressive HNSCC a treatment suitable for aggressive HNSCC.

In still another aspect, the present disclosure encompasses a kit for determining the aggressiveness of head and neck squamous cell carcinoma (HNSCC) in a subject. The kit comprises a substrate for holding a test sample isolated from the subject; an at least 10-nucleic acid molecular signature disclosed herein; agents for detection/measurement of the at least 10-nucleic acid molecular signature; and optionally, printed instructions for reacting the agents with the biological sample or a portion of the biological sample to detect the presence or amount of each nucleic acid of the at least 10-nucleic acid molecular signature in the biological sample.

### BRIEF DESCRIPTION OF THE FIGURES

The application file contains at least one drawing executed in color. Copies of this patent application publication with color drawing(s) will be provided by the Office upon request and payment of the necessary fee.

FIG. 1 depicts images and graphs of next generation sequencing analysis of MOC cell lines. (A) Overview of MOC cell line model illustrating biologic behavior upon flank injection of cells. Note MOC23 (italicized) only grows in RAG2^{-/-} mice. (B) Number of SNVs in each MOC line. (C) Distribution of DMBA induced changes in 6 core driver pathways of HNSCC shown as a “signal flag” plot of mutation subtypes in the indolent and aggressive cell lines (numbers of genes in each driver pathway in parentheses). The boxed nucleotide changes (A→T, T→A, C→A and G→T) represent the most common DMBA induced alterations. Note, the underlined G:C→T:A change is typical for tobacco-induced mutations ¹¹. (D) Selected Oncoprint of mutation rates within the TCGA cohort for AKAP proteins showing that 57/279 patients have alterations in indicated AKAPs. (E) Oncoprint for MED proteins showing that 41/279 patients have alterations in indicated MED components.

FIG. 2 depicts a graph of the average statistics for depth of coverage at 20×, 30× and 40× of all MOC lines. Robust reads were obtained in all samples with a range of 97-98% for 20×, 93-95% for 30× and 88-92% for 40×.

FIG. 3 depicts a graph of oncoprints from cBio of AKAP9, MED12L, THSD7A, MUC5B, MYH6, LAMA1, LRP2, and 3 RAS (Table 9) genes representing other candidate tumor promoters as compared to the 3 RAS genes. Note that for AKAPs and MED components, the inventors found 9 AKAP family member mutations in 20.4% of tumors, with AKAP9 changes in 7% (FIG. 1D). Six components of the mediator complex were mutated in 14.7% of cases, with MEDI 2L changes in 5% (FIG. 1E).

FIG. 4 depicts graphs of the number of nsSNV's per node negative (N0) and node positive (N₊) tumor in (A) all TCGA OSCC (note that patient TCGA-D6-6516 (N0) with 1463 mutations is not included in this graph) and (B) TCGA OSCC patients who had smoking history reported. There was no significant difference in the average number of mutations between N0 (3272 nsSNVs) and N₊ (3097 nsSNVs) patients regardless of smoking status (Tables 11, 12). This analysis showed 17 genes commonly mutated in mouse indolent and human N0 tumors and 55 common genes mutated in mouse aggressive and human N₊tumors (Table 13). However, none of these common genes were mutated at high frequency in the human N0 or N₊ datasets (Table 14 and 15). Finally, comparing N0 and N₊ tumors from human TCGA data also showed that specific mutations occur infrequently in both the metastatic and non-metastatic tumors (data not shown).

FIG. 5A depicts Principal component analysis (PCA) of MOC lines shows clustering of MOC1 and MOC22 near oral keratinocytes (OK). MOC23 is separated from all lines whereas the related aggressive lines all cluster together. FIG. 5B depicts Heatmap of aggressive growth signature. Unsupervised clustering of microarray data reveals a mouse signature of metastasis. FIG. 5C depicts microarray values (MA) (left) and qRT-PCR analysis (Taqman) (right) for Nkx2-3 and Foxal in MOC1 (indolent) and MOC2-10 (aggressive) lines showing dramatic upregulation in the aggressive line. FIG. 5D depicts Microarray values (MA) (left) and qRT-PCR analysis (Taqman) (right) comparing Hoxb7 and Bmp4 expression in MOC2 (LN-lymph node metastatic) and MOC2-10 (LN/lung metastatic). FIG. 5E depicts Kaplan-Meier analysis of OCAMP-A on UW/FHCRC dataset showing significant survival difference based on enrichment of mouse metastasis signature (p<0.01). FIG. 5F and FIG. 5G depict GSEA plots showing significant enrichment of both (FIG. 5F) up and (FIG. 5G) down OCAMP-A transcripts in the UW/FHCRC 97-patient OSCC dataset (p<0.05). FIG. 5H and FIG. 5I depict GSEA plots showing significant enrichment of both (FIG. 5H) up and (FIG. 5I) down OCAMP-A transcripts in the 134 OSCC patients from the TCGA dataset (p<0.001). FIG. 5J and FIG. 5K depicts GSEA plots showing enrichment of both (FIG. 5J) up and (FIG. 5K) down OCAMP-A transcripts in the 71 OSCC patients from the MD Anderson dataset (p=0.57, n.s.=not significant).

FIG. 6 depicts a graph of a SAM plotsheet of MOC line microarray data with estimated miss rates for delta=4.68.

FIG. 7A and FIG. 7B depict GSEA data showing significant enrichment of both (FIG. 7A) up and (FIG. 7B) down OCAMP-A transcripts on UW/FHCRC data. FIG. 7C and FIG. 7D depict the first condensation of OCAMP-A on UW/FHCRC data of (FIG. 7C) up enriched nucleic acids and (FIG. 7D) down enriched nucleic acids. FIG. 7E and FIG. 7F depict GSEA data showing significant enrichment of both (FIG. 7E) up and (FIG. 7F) down OCAMP-A transcripts on MD data. FIG. 7G and FIG. 7H depict the first condensation of OCAMP-A on MD data of (FIG. 7G) up enriched nucleic acids and (FIG. 7H) down enriched nucleic acids.

FIG. 8A depicts a schematic of iterative GSEA showing selection of enrichment in each dataset (1^{st} trim) and tandem enrichment in a 2^{nd} trim that finally yields the 118-nucleic acid OCAMP-B signature. FIG. 8B depicts an illustration of 1^{st} trim on up transcripts in TCGA data shown in FIG. 5H. FIG. 8C depicts an illustration of 1^{st} trim on down transcripts in TCGA data shown in FIG. 51. FIG. 8D depicts a Venn diagram of OCAMP-A enrichment on three datasets with 118 common nucleic acids defined as OCAMP-B. Note that 56 OCAMP-A nucleic acids did not enrich in any dataset. FIG. 8E depicts down transcripts in TCGA dataset. FIG. 8F depicts down transcripts in UW/FHCRC dataset. FIG. 8G depicts down transcripts in MD dataset. FIG. 8H depicts up transcripts in TCGA dataset. FIG. 81 depicts up transcripts in UW/FHCRC dataset. FIG. 8J depicts up transcripts in MD dataset. FIG. 8K depicts Kaplan-Meier analysis after OCAMP-B based weighted voting of MD dataset showing significant survival difference (p<0.001).

FIG. 9 depicts an illustration and graphs of a clinical assay for genetic stratification of OSCCs. (A) Schematic illustrating the selection of 42 OCAMP-A nucleic acids and SVM processing on training set samples to identify the best discriminating nucleic acids. (B) An independent UPENN dataset is classified with high accuracy (21/22 tumors) by OCAMP-B weighted voting (WV output) with respect to lymph node metastatic status (Path=known pathologic status). (C) Discriminant scores from SVM analysis showing successful stratification in 12/13 FFPE and 17/18 fresh biopsy test cases of metastatic nodal disease using a qRT-PCR assay.

FIG. 10 depicts graphs showing that OCAMP-B augments stage based survival prediction (A) and OCAMP-B improves clinical node stage (B). (A) Percent cumulative survival by stage compared to signature-based assignment (patent numbers are within bars). (B) OCAMP assignment and pathologic node status are equivalent 18/18 patients who were cN₀ but were pN₊ were correctly identified.

FIG. 11 depicts a graph of disease specific survival (DSS) after weighted voting classification of OCAMP-B signature on the MD Anderson dataset that shows worse outcome for those with aggressive classification (p=0.028).

FIG. 12 depicts graphs of (A) disease specific survival (DSS) and (B) overall survival (OS) after weighted voting classification of OCAMP-B signature on the UW/FHCRC dataset that shows worse outcome for those with aggressive classification (p<0.01).

### DETAILED DESCRIPTION OF THE INVENTION

The present invention provides methods and kits to detect aggressiveness of head and neck squamous cell carcinoma (HNSCC), specifically oral squamous cell carcinoma (OSCC), using a molecular signature. The molecular signature may allow a more accurate diagnosis or prognosis of HNSCC, specifically OSCC, in a subject. Furthermore, the molecular signature may allow for optimal treatment of a subject in need thereof.

### I. Molecular Signature to Determine the Aggressiveness of HNSCC

One aspect of the present invention provides a method to determine the aggressiveness of head and neck squamous cell carcinoma (HNSCC) in a subject. The method comprises providing a test sample from a subject known to have HNSCC, determining the nucleic acid expression levels in the test sample of at least a 10-nucleic acid molecular signature, comparing these expression levels to reference expression levels of the nucleic acids of the molecular signature, wherein differentially expressed levels in the test sample compared to the reference expression levels indicates aggressiveness. In a specific embodiment, the HNSCC is OSCC.

The term “molecular signature” used herein refers to a set of nucleic acids that are differentially expressed in a subject. For example, with respect to OSCC, the molecular signature may be differentially expressed in a subject according to the aggressiveness of OSCC and thus may be predictive of prognosis, metastasis potential and the benefit of adjuvant chemotherapy. In one embodiment, the molecular signature is a 10-nucleic acid molecular signature consisting of DSG3, IGF2BP1, MUC1, EOMES, NKX2-3, FOXA1, DMKN, GSTA4, ANKRD1, and KLF2. In another embodiment, the molecular signature is a 19-nucleic acid molecular signature consisting of BEX2, DSG3, HOXB7, IGF2BP1, MUC1, EOMES, NKX2-3, MEIS1, UNC13B, TDRKH, FNTA, FOXA1, DMKN, GSTA4, IVL, ANKRD1, GSPT2, KLF2, and LPAR1. Alternatively, a molecular signature of the invention may comprise 10 to 20, 20 to 30, 30 to 50, 50 to 100, 100 to 200, 200 to 300, 300 to 400 and more than 400 nucleic acids. In one embodiment, a nucleic acid signature of the invention may comprise at least 10, at least 11, at least 12, at least 13, at least 14, at least 15, at least 16, at least 17, at least 18, at least 19, or at least 20 nucleic acids from Table A. In another embodiment, a nucleic acid signature of the invention may comprise at least 20, at least 21, at least 22, at least 23, at least 24, at least 25, at least 26, at least 27, at least 28, at least 29, or at least 30 nucleic acids from Table A. In still another embodiment, a nucleic acid signature of the invention may comprise at least 30, at least 31, at least 32, at least 33, at least 34, at least 35, at least 36, at least 37, at least 38, at least 39, or at least 40 nucleic acids from Table A. In still yet another embodiment, a nucleic acid signature of the invention may comprise at least 40, at least 41, at least 42, at least 43, at least 44, at least 45, at least 46, at least 47, at least 48, at least 49, or at least 50 nucleic acids from Table A. In a different embodiment, a nucleic acid signature of the invention may comprise at least 50, at least 51, at least 52, at least 53, at least 54, at least 55, at least 56, at least 57, at least 58, at least 59, or at least 60 nucleic acids from Table A. In certain embodiments, a nucleic acid signature of the invention may comprise at least 60, at least 61, at least 62, at least 63, at least 64, at least 65, at least 66, at least 67, at least 68, at least 69, or at least 70 nucleic acids from Table A. In other embodiments, a nucleic acid signature of the invention may comprise at least 70, at least 71, at least 72, at least 73, at least 74, at least 75, at least 76, at least 77, at least 78, at least 79, or at least 80 nucleic acids from Table A. In different embodiments, a nucleic acid signature of the invention may comprise at least 80, at least 81, at least 82, at least 83, at least 84, at least 85, at least 86, at least 87, at least 88, at least 89, or at least 90 nucleic acids from Table A. In another embodiment, a nucleic acid signature of the invention may comprise at least 90, at least 91, or 92 nucleic acids from Table A. Nucleic acids have transcript variants due to alternative splicing. A skilled artisan would be able to determine various transcript variants from the accession numbers provided.

TABLE A
Nucleic acids for Molecular Signature

| | | | Mus musculus | Homo sapiens |
|---|---|---|---|---|
| | Nucleic | | Accession | Accession |
| | acid | Nucleic acid name | Number | Number |
| 1 | BEX2 | Brain expressed X-linked 2 | NM_009749.2 | NM_001168399.1 |
| 2 | DSG3 | Desmoglein 3 | NM_030596.3 | NM_001944.2 |
| 3 | HOXB7 | Homeobox B7 | NM_010460.2 | NM_004502.3 |
| 4 | IGF2BP1 | Insulin-like growth factor 2 mRNA binding | NM_009951.4 | NM_006546.3 |
| | | protein 1 | | |
| 5 | MUC1 | Mucin 1, cell surface associated | NM_013605.2 | NM_002456.5 |
| 6 | EOMES | Eomesodermin | NM_010136.3 | NM_001278182.1 |
| 7 | NKX2-3 | NK2 homeobox 3 | NM_008699.2 | NM_145285.2 |
| 8 | MEIS1 | Meis homeobox 1 | NM_010789.3 | NM_002398.2 |
| 9 | UNC13B | Unc-13 homolog B | NM_021468.2 | NM_006377.3 |
| 10 | TDRKH | Tudor and KH domain-containing protein | NM_028307.1 | NM_001083965.1 |
| 11 | FNTA | Farnesyltransferase/geranylgeranyltransferase | NM_008033.3 | NM_002027.2 |
| | | type-1 subunit alpha | | |
| 12 | FOXA1 | Forkhead box protein A1 | NM_008259.3 | NM_004496.3 |
| 13 | DMKN | Dermokine | NM_028618.2 | NM_001035516.3 |
| 14 | GSTA4 | Glutathione S-transferase A4 | NM_010357.3 | NM_001512.3 |
| 15 | IVL | Involucrin | NM_008412.3 | NM_005547.2 |
| 16 | ANKRD1 | Ankyrin repeat domain-containing protein 1 | NM_013468.3 | NM_014391.2 |
| 17 | GSPT2 | G1 to S phase transition 2 | NM_008179.2 | NM_018094.4 |
| 18 | KLF2 | Kruppel-like Factor 2 | NM_008452.2 | NM_016270.2 |
| 19 | LPAR1 | Lysophosphatidic acid receptor 1 | NM_010336.2 | NM_001401.3 |
| 20 | GPX2 | Glutathione peroxidase 2 | NM_030677.2 | NM_002083.3 |
| 21 | MGRN1 | Mahogunin ring finger 1, E3 ubiquitin | NM_001252437.1 | NM_015246.3 |
| | | protein ligase | | |
| 22 | PCYT1B | Phosphate cytidylyltransferase 1, choline, | NM_211138.1 | NM_004845.4 |
| | | beta | | |
| 23 | WRB | Tryptophan rich basic protein | NM_207301.2 | NM_004627.4 |
| 24 | TRIM39 | Tripartite motif containing 39 | NM_024468. | NM_021253.3 |
| 25 | IL18RAP | Interleukin 18 receptor accessory protein | NM_010553.4 | NM_003853.3 |
| 26 | P4HA2 | Prolyl 4-hydroxylase, alpha polypeptide II | NM_001136076.2 | NM_004199.2 |
| 27 | RAB38 | Member RAS oncogene family | NM_028238.7 | NM_022337.2 |
| 28 | GSTO2 | Glutathione S-transferase omega 2 | NM_026619.2 | NM_183239.1 |
| 29 | SART1 | Squamous cell carcinoma antigen | NM_016882.3 | NM_005146.4 |
| | | recognized by T cells | | |
| 30 | INSM1 | Insulinoma-associated 1 | NM_016889.3 | NM_002196.2 |
| 31 | STARD13 | StAR-related lipid transfer (START) | NM_001163493.1 | NM_178006.3 |
| | | domain containing 13 | | |
| 32 | MLLT11 | Myeloid/lymphoid or mixed-lineage | NM_019914.4 | NM_006818.3 |
| | | leukemia (trithorax homolog, Drosophila); | | |
| | | translocated to, 11 | | |
| 33 | DISP1 | Dispatched homolog 1 (Drosophila) | NM_026866.3 | NM_032890.3 |
| 34 | LYNX1 | Ly6/neurotoxin 1 | NM_011838.4 | NM_023946.3 |
| 35 | RAB40C | Member RAS oncogene family | NM_139154.2 | NM_001172663.1 |
| 36 | SYTL1 | Synaptotagmin-like 1 | NM_031393.2 | NM_001193308.1 |
| 37 | PGPEP1 | Pyroglutamyl-peptidase I | NM_023217.4 | NM_017712.2 |
| 38 | FMNL1 | Formin-like 1 | NM_019679.2 | NM_005892.3 |
| 39 | ADPRHL2 | ADP-ribosylhydrolase like 2 | NM_133883.2 | NM_017825.2 |
| 40 | CACNB2 | Calcium channel, voltage-dependent, beta | NM_023116.4 | NM_000724.3 |
| | | 2 subunit | | |
| 41 | MAGEE1 | Melanoma antigen family E1 | NM_053201.3 | NM_020932.2 |
| 42 | CDH2 | Cadherin 2, type 1, N-cadherin (neuronal) | NM_007664.4 | NM_001792.3 |
| 43 | CELF2 | CUGBP, Elav-like family member 2 | NM_001110228.1 | NM_001025076.2 |
| 44 | CRK | V-crk avian sarcoma virus CT10 oncogene | NM_001277219.1 | NM_005206.4 |
| | | homolog | | |
| 45 | HR | Hair growth associated | NM_021877.3 | NM_005144.4 |
| 46 | FAHD2A | Fumarylacetoacetate hydrolase domain | NM_029629.2 | NM_016044.2 |
| | | containing 2A | | |
| 47 | E2F4 | E2F transcription factor 4, p107/p130- | NM_148952.1 | NM_001950.3 |
| | | binding | | |
| 48 | PIP5K1A | Phosphatidylinositol-4-phosphate 5-kinase, | NM_001293707.1 | NM_001135638.1 |
| | | type I, alpha | | |
| 49 | DCBLD2 | Discoidin, CUB and LCCL domain | NM_028523.3 | NM_080927.3 |
| | | containing 2 | | |
| 50 | CASP1 | Caspase 1, apoptosis-related cysteine | NM_009807.2 | NM_001257118.2 |
| | | peptidase | | |
| 51 | SYTL4 | Synaptotagmin-like 4 | NM_013757.2 | NM_080737.2 |
| 52 | TACSTD2 | Tumor-associated calcium signal | NM_020047.3 | NM_002353.2 |
| | | transducer 2 | | |
| 53 | PDGFA | Platelet-derived growth factor alpha | NM_008808.3 | NM_002607.5 |
| | | polypeptide | | |
| 54 | TIMP2 | TIMP metallopeptidase inhibitor 2 | NM_011594.3 | NM_003255.4 |
| 55 | CAPN5 | Calpain 5 | NM_007602.4 | NM_004055.4 |
| 56 | SIRT5 | Sirtuin 5 | NM_178848.3 | NM_012241.4 |
| 57 | TRAFD1 | TRAF-type zinc finger domain containing 1 | NM_001163470.1 | NM_001143906.1 |
| 58 | COLGALT1 | Collagen beta(1-O)galactosyltransferase 1 | NM_146211.3 | NM_024656.2 |
| 59 | ME2 | Malic enzyme 2, NAD(+)-dependent, | NM_145494.2 | NM_002396.4 |
| | | mitochondrial | | |
| 60 | PLA2G15 | Phospholipase A2, group XV | NM_133792.2 | NM_012320.3 |
| 61 | BBS4 | Bardet-Biedl syndrome 4 | NM_175325.3 | NM_033028.4 |
| 62 | RAB3D | Member RAS oncogene family | NM_031874.4 | NM_004283.3 |
| 63 | TAF13 | TAF13 RNA polymerase II, TATA box | NM_025444.2 | NM_005645.3 |
| | | binding protein (TBP)-associated factor, | | |
| | | 18kDa | | |
| 64 | FARP1 | FERM, RhoGEF (ARHGEF) and pleckstrin | NM_134082.3 | NM_005766.3 |
| | | domain protein 1 (chondrocyte-derived) | | |
| 65 | LASP1 | LIM and SH3 protein 1 | NM_010688.4 | NM_006148.3 |
| 66 | PCOLCE2 | Procollagen C-endopeptidase enhancer 2 | NM_029620.2 | NM_013363.3 |
| 67 | EPHA1 | EPH receptor A1 | NM_023580.4 | NM_005232.4 |
| 68 | FXYD3 | FXYD domain containing ion transport | NM_008557.2 | NM_005971.3 |
| | | regulator 3 | | |
| 69 | ECM1 | Extracellular matrix protein 1 | NM_007899.2 | NM_004425.3 |
| 70 | PKIA | Protein kinase (cAMP-dependent, | NM_008862.3 | NM_006823.3 |
| | | catalytic) inhibitor alpha | | |
| 71 | RGL2 | Ral guanine nucleotide dissociation | NM_009059.2 | NM_004761.4 |
| | | stimulator-like 2 | | |
| 72 | CYR61 | Cysteine-rich, angiogenic inducer, 61 | NM_010516.2 | NM_001554.4 |
| 73 | VDR | Vitamin D (1,25- dihydroxyvitamin D3) | NM_009504.4 | NM_000376.2 |
| | | receptor | | |
| 74 | STXBP1 | Syntaxin binding protein 1 | NM_001113569.1 | NM_003165.3 |
| 75 | P2RY1 | Purinergic receptor P2Y, G-protein | NM_008772.5 | NM_002563.3 |
| | | coupled, 1 | | |
| 76 | OLFML2B | Olfactomedin-like 2B | NM_177068.4 | NM_001297713.1 |
| 77 | PPFIBP2 | PTPRF interacting protein, binding protein | NM_008905.2 | NM_003621.3 |
| | | 2 (liprin beta 2) | | |
| 78 | TIAM1 | T-cell lymphoma invasion and metastasis 1 | NM_009384.3 | NM_003253.2 |
| 79 | AP1M1 | Adaptor-related protein complex 1, mu 1 | NM_007456.4 | NM_001130524.1 |
| | | subunit | | |
| 80 | STARD5 | StAR-related lipid transfer (START) | NM_023377.4 | NM_181900.2 |
| | | domain containing 5 | | |
| 81 | SLC6A9 | Solute carrier family 6 (neurotransmitter | NM_008135.4 | NM_006934.3 |
| | | transporter, glycine), member 9 | | |
| 82 | MTMR9 | Myotubularin related protein 9 | NM_177594.1 | NM_015458.3 |
| 83 | EPHX1 | Epoxide hydrolase 1, microsomal | NM_010145.2 | NM_000120.3 |
| | | (xenobiotic) | | |
| 84 | AQP3 | Aquaporin 3 (Gill blood group) | NM_016689.2 | NM_004925.4 |
| 85 | PI4KA | Phosphatidylinositol 4-kinase, catalytic, | NM_001001983.2 | NM_058004.3 |
| | | alpha | | |
| 86 | WNT4 | Wingless-type MMTV integration site | NM_009523.2 | NM_030761.4 |
| | | family, member 4 | | |
| 87 | DHX38 | DEAH (Asp-Glu-Ala-His) box polypeptide | NM_178380.1 | NM_014003.3 |
| | | 38 | | |
| 88 | ASS1 | Argininosuccinate synthase 1 | NM_007494.3 | NM_000050.4 |
| 89 | SLPI | Secretory leukocyte peptidase inhibitor | NM_011414.3 | NM_003064.3 |
| 90 | IMPA2 | Inositol(myo)-1(or 4)-monophosphatase 2 | NM_053261.2 | NM_014214.2 |
| 91 | TNNC1 | Troponin C type 1 (slow) | NM_009393.2 | NM_003280.2 |
| 92 | CBR3 | Carbonyl reductase 3 | NM_173047.3 | NM_001236.3 |

Additionally, it is realized that the molecular signature may further comprise one or more nucleic acids from Table B. For example, the molecular signature may further comprise 1 to 10, 10 to 20, 20 to 30, 30 to 50, 50 to 100, 100 to 200, 200 to 300, 300 to 390 nucleic acids from Table B. In an embodiment, the molecular signature may further comprise 1 to 10 nucleic acids from Table B. In another embodiment, the molecular signature may further comprise 10 to 20 nucleic acids from Table B. In still another embodiment, the molecular signature may further comprise 20 to 30 nucleic acids from Table B. In still yet another embodiment, the molecular signature may further comprise 30 to 50 nucleic acids from Table B. In a different embodiment, the molecular signature may further comprise 50 to 100 nucleic acids from Table B. In certain embodiments, the molecular signature may further comprise 100 to 200 nucleic acids from Table B. In other embodiments, the molecular signature may further comprise 200 to 300 nucleic acids from Table B. In a further embodiment, the molecular signature may further comprise 300 to 390 nucleic acids from Table B. In addition, other nucleic acids not herein described may be combined with any of the presently disclosed nucleic acids to aid in the determination of the aggressiveness of HNSCC, specifically OSCC.

For Table B, common nucleic acid names listed for the nucleic acids are known in the art. A skilled artisan would be able to determine the common nucleic acid names and the various sequences of the nucleic acids listed similar to the information provided in Table A.

TABLE B
Potential additional nucleic acids for aggressiveness molecular signature

| | | | | | |
|---|---|---|---|---|---|
| PCOLCE | MKRN3 | GIT1 | STK32C | PHLDA3 | COL12A1 |
| PACSIN2 | LSM11 | SOCS7 | SLC35F2 | PCBP3 | ECE1 |
| CTGF | BCAR1 | IGSF11 | SETD6 | HES1 | THBS2 |
| FGF13 | GCA | TRMT2A | RRAGB | CEBPB | COL16A1 |
| SP8 | IQGAP3 | CAMKK1 | DST | TEAD3 | MET |
| TNPO2 | MSH6 | GSG1 | PTPN21 | SLC1A3 | TNFRSF1A |
| NEK8 | UCK2 | CHN2 | LRP11 | XDH | CLSTN1 |
| RAD23A | EXOC8 | FLII | NISCH | CELSR2 | SOSTDC1 |
| ELL | HAVCR2 | TPD52L1 | CDC42BPB | SNCG | ART4 |
| XPO4 | PDP1 | ASL | PLCB3 | BCAP31 | FZD7 |
| ZFPM2 | FOSL1 | LTK | DNMBP | CD9 | OCIAD2 |
| AKAP12 | DEPDC1B | CCDC109A | SPIRE2 | ATP1A1 | HS6ST1 |
| RPP21 | B3GNT5 | CAMK2B | NADSYN1 | TEF | FST |
| PRMT5 | TPH1 | DDX49 | UNC13D | MMP2 | APP |
| USP13 | ESPL1 | DNAJA2 | DHX32 | MCAT | CLOCK |
| PLCL2 | UPF3A | SYN1 | PRKCZ | COL18A1 | PPP1R14C |
| RBPMS | GMIP | CLCF1 | PLEKHA2 | RHOJ | DLL1 |
| PKN1 | USP43 | PHACTR4 | GM2A | RBMS3 | KIF13A |
| ZFP57 | HIST1H2AE | NOB1 | CHCHD7 | CASQ2 | FJX1 |
| DDAH1 | PCSK9 | LMTK2 | TBX3 | IFT140 | PORCN |
| RIMS2 | WDR6 | PPAP2A | TMEM108 | STAB1 | LAPTM4A |
| UCHL1 | CDC23 | MIA2 | AK1 | LRP1 | KCMF1 |
| ARHGEF18 | DONSON | GFOD1 | OSGEP | AMPD1 | ODZ4 |
| NEO1 | HHEX | NSF | RENBP | SLC39A4 | JAG2 |
| COG1 | TMEM20 | GPS2 | SERPINF1 | RAB15 | SALE |
| RAB3B | VTI1A | MY01C | NPHP4 | IFITM2 | ITGB4 |
| KCNF1 | FUT10 | NUDCD3 | UGT1A10 | LSP1 | SLC7A8 |
| DUSP3 | PTTG1 | TACC1 | PTN | PPBP | PPP5C |
| CRYL1 | MARVELD3 | PPA2 | PLD3 | ORM1 | MY01B |
| CKB | HLCS | NT5C2 | RFX2 | PTMS | EFS |
| CGNL1 | FKBP5 | LRSAM1 | RGMA | APOBEC1 | TAPBP |
| FGD3 | DCAF5 | TRAPPC5 | ADA | SLC5A8 | BNC1 |
| EGLN1 | PPM1D | PRDX2 | ARHGEF19 | DACT2 | BCL7A |
| GPRC5A | PLSCR2 | TJP1 | PBX1 | GSTO1 | INHBB |
| RPS6KL1 | IGF2BP2 | PRODH | TMEM53 | POLR2J | SSFA2 |
| TMEM160 | ARHGAP8 | MTCH2 | NXN | DCXR | IMPACT |
| PDSS1 | MOV10 | ZFPM1 | SCMH1 | VAMP5 | NRTN |
| RIN3 | TDRD7 | EPN2 | NKD2 | PSTPIP1 | FSCN1 |
| RBPMS2 | PBX4 | HNF1B | ROR2 | CYP2S1 | BCL6 |
| GAN | SFXN1 | SCAMP5 | PTPRU | PIGF | ING4 |
| ZDHHC3 | ISG20 | IKBKB | PLXNA2 | CTH | SCP2 |
| GIPC2 | POU4F1 | GALK1 | KLF4 | TSPO | PER2 |
| OXSR1 | KCTD9 | ADK | IFITM3 | NDUFA4 | SLC11A2 |
| TUBB2B | CLCA4 | THYN1 | PPP1CB | MOXD1 | KCTD15 |
| LEPRE1 | FAIM | B3GNT3 | COL23A1 | WTIP | GALNTL4 |
| GLT25D1 | NUP210 | VGF | GYLTL1B | ARF5 | ATP1OD |
| NUP133 | TJP3 | MSRB2 | MST1R | RNASET2 | SSBP2 |
| EPS8 | PKP2 | ATP6V0A1 | SULF1 | PRICKLE3 | LIMK2 |
| GJC1 | FGF5 | FGF22 | ATG5 | CCND2 | SOX15 |
| SF3B2 | PEA15 | HCN2 | EXT1 | GPR108 | IRX5 |
| GALNT10 | COL4A2 | SERPIND1 | DAPK1 | RSPH1 | SP6 |
| CHERP | DCLK2 | ABHD14B | PPFIBP1 | ATOX1 | PARD6G |
| CSTF3 | FHOD1 | SAMD10 | CDC42EP3 | SEMA4A | TAPBPL |
| GSTCD | SLC44A4 | BSPRY | VGLL4 | MGST2 | PVRL1 |
| RAB21 | SMG5 | WDR33 | M6PR | HYAL1 | CXCL14 |
| HSD17B7 | CHRNB1 | ANKRD50 | ICAM1 | FXYD4 | GNA15 |
| DUSP11 | CLDN6 | DPP3 | FBXO32 | B3GALT4 | TMEM54 |
| FAS | PRDX4 | F2R | INPP5F | IL17RE | VSNL1 |
| PSMB9 | PPP1R9A | COL4A1 | TNFAIP8 | PLCH2 | RAPGEFL1 |
| MYH10 | LAMB2 | ZCCHC14 | SIK1 | GSTK1 | PGAP2 |
| AQP8 | SPRY3 | SCAMP3 | ST5 | IL171RC | CRABP2 |
| UNC5B | BACE1 | NANOS1 | SORL1 | RARG | TRIM29 |
| RASL11A | PVR | MED10 | OSMR | CSTB | ADORA2B |
| LRP5 | APBB1 | BTBD12 | LY6E | RBAK | GPR64 |
| F2RL1 | ULK1 | CDH6 | SEC61B | IRAK2 | RNF19A |

The molecular signature may further comprise one or more nucleic acid used as a normalization control. A normalization control compensates for systematic technical differences between experiments, to see more clearly the systematic biological differences between samples. A normalization control is a nucleic acid whose expression is not expected to be different across samples. Generally, these nucleic acids may be known as ‘housekeeping’ nucleic acids which are required for basic cell processes. Non-limiting examples of housekeeping nucleic acids commonly used as normalization controls include GAPD, ACTB, B2M, TUBA, G6PD, LDHA, HPRT, ALDOA, PFKP, PGK1, PGAM1, VIM and UBC. In a specific embodiment, the nucleic acid used as a normalization control is one or more nucleic acids selected from the group consisting of UBC, GAPDH and actin.

The method includes determining the nucleic acid expression level of each nucleic acid of the molecular signature. The term “level of expression” or “expression level” as used herein refers to a measurable level of expression of the nucleic acids, such as, without limitation, the level of messenger RNA transcript expressed or a specific exon or other portion of a transcript, the level of proteins or portions thereof expressed from the nucleic acids, the number or presence of DNA polymorphisms of the nucleic acids, the enzymatic or other activities of the nucleic acids, and the level of a specific metabolite. The term “nucleic acid” includes DNA and RNA and can be either double stranded or single stranded. In a specific embodiment, determining the level of expression of a nucleic acid of the molecular signature comprises, in part, measuring the level of RNA expression. The term “RNA” includes mRNA transcripts, and/or specific spliced or other alternative variants of mRNA, including anti-sense products. The term “RNA product of the nucleic acid” as used herein refers to RNA transcripts transcribed from the nucleic acids and/or specific spliced or alternative variants. Non-limiting examples of suitable methods to assess a nucleic acid expression level may include arrays, such as microarrays, PCR, such as RT-PCR (including quantitative RT-PCR), nuclease protection assays and Northern blot analyses.

In one embodiment, the nucleic acid expression levels are determined by using an array, such as a microarray. For example, a plurality of nucleic acid probes that are complementary or hybridizable to an expression product of each nucleic acid of the molecular signature are used on the array. Accordingly, 10 to 20, 20 to 30, 30 to 50, 50 to 100, 100 to 200, 200 to 300, 300 to 400 and more than 400 nucleic acids may be used on the array. The term “hybridize” or “hybridizable” refers to the sequence specific non-covalent binding interaction with a complementary nucleic acid. In a preferred embodiment, the hybridization is under high stringency conditions. Appropriate stringency conditions which promote hybridization are known to those skilled in the art, or can be found in Current Protocols in Molecular Biology, John Wiley & Sons, N.Y. (1989), 6.3.1 6.3.6. The term “probe” as used herein refers to a nucleic acid sequence that will hybridize to a nucleic acid target sequence. In one example, the probe hybridizes to an RNA product of the nucleic acid or a nucleic acid sequence complementary thereof. The length of probe depends on the hybridization conditions and the sequences of the probe and nucleic acid target sequence. In one embodiment, the probe is at least 8, 10, 15, 20, 25, 50, 75, 100, 150, 200, 250, 400, 500 or more nucleotides in length.

In another embodiment, the nucleic acid expression levels may be determined using PCR. Methods of PCR are well and widely known in the art, and may include quantitative PCR, semi-quantitative PCR, multiplex PCR, or any combination thereof. Specifically, the amount of nucleic acid expression may be determined using quantitative RT-PCR. Methods of performing quantitative RT-PCR are common in the art. In such an embodiment, the primers used for quantitative RT-PCR may comprise a forward and reverse primer for each nucleic acid of the molecular signature. The term “primer” as used herein refers to a nucleic acid sequence, whether occurring naturally as in a purified restriction digest or produced synthetically, which is capable of acting as a point of synthesis when placed under conditions in which synthesis of a primer extension product, which is complementary to a nucleic acid strand is induced (e.g. in the presence of nucleotides and an inducing agent such as DNA polymerase and at a suitable temperature and pH). The primer must be sufficiently long to prime the synthesis of the desired extension product in the presence of the inducing agent. The exact length of the primer will depend upon factors, including temperature, sequences of the primer and the methods used. A primer typically contains 15-25 or more nucleotides, although it can contain less or more. The factors involved in determining the appropriate length of primer are readily known to one of ordinary skill in the art.

The nucleic acid expression level may be measured by measuring an entire mRNA transcript for a nucleic acid of the molecular signature, or measuring a portion of the mRNA transcript for a nucleic acid of the molecular signature. For instance, if a nucleic acid array is utilized to measure the level of mRNA expression, the array may comprise a probe for a portion of the mRNA of the nucleic acid of the molecular signature, or the array may comprise a probe for the full mRNA of the nucleic acid sequence of the molecular signature. Similarly, in a PCR reaction, the primers may be designed to amplify the entire cDNA sequence of the nucleic acid of the molecular signature, or a portion of the cDNA sequence. One of skill in the art will recognize that there is more than one set of primers that may be used to amplify either the entire cDNA or a portion of the cDNA for a nucleic acid of the molecular signature. Methods of designing primers are known in the art. Methods of extracting RNA from a test sample are known in the art.

The level of expression of each nucleic acid of the molecular signature may be compared to a reference expression level for each nucleic acid of the molecular signature. The subject expression levels of the nucleic acids in the molecular signature in a test sample are compared to the corresponding reference expression levels of the nucleic acids of the molecular signature to determine aggressiveness or predict prognosis. Accordingly, a reference expression level may comprise 10 to 20, 20 to 30, 30 to 50, 50 to 100, 100 to 200, 200 to 300, 300 to 400 and more than 400 expression levels based on the number of nucleic acids in the molecular signature. Any suitable reference expression level known in the art may be used. For example, a suitable reference expression level may be the level of molecular signature in a test sample obtained from a subject or group of subjects of the same species that have no signs or symptoms of HNSCC. In another example, a suitable reference expression level may be the level of molecular signature in a test sample obtained from a subject or group of subjects of the same species that have not been diagnosed with HNSCC. In still another example, a suitable reference expression level may be the level of molecular signature in a test sample obtained from a subject or group of subjects of the same species that have signs or symptoms of HNSCC. In yet still another example, a suitable reference expression level may be the level of molecular signature in a test sample obtained from a subject or group of subjects of the same species that been diagnosed with HNSCC. In a different example, a suitable reference expression level may be the level of molecular signature in a test sample obtained from a subject or group of subjects of the same species that have indolent HNSCC. In a different example, a suitable reference expression level may be the level of molecular signature in a test sample obtained from a subject or group of subjects of the same species that have aggressive HNSCC. In a different example, a suitable reference expression level may be the background signal of the assay as determined by methods known in the art. In another example, a suitable reference expression level may be a measurement of the molecular signature in a reference sample obtained from the same subject. The reference sample comprises the same type of biological sample as the test sample, and may or may not have been obtained from the subject when HNSCC was not suspected. A skilled artisan will appreciate that that is not always possible or desirable to obtain a reference sample from a subject when the subject is otherwise healthy. For example, in an acute setting, a reference sample may be the first sample obtained from the subject at presentation. In another example, when monitoring effectiveness of a therapy, a reference sample may be a sample obtained from a subject before therapy began. In a specific embodiment, a reference expression level may be the level of expression of each nucleic acid of the molecular signature in subjects that have indolent OSCC. Such a reference expression level may be used to create a control value that is used in testing samples from new subjects. In such an embodiment, the “control” is a predetermined value for each nucleic acid of the molecular signature.

The expression level of each nucleic acid of the molecular signature is compared to the reference expression level of each nucleic acid of the molecular signature to determine if the nucleic acids of the molecular signature in the test sample are differentially expressed relative to the reference expression level of the corresponding nucleic acid. The term “differentially expressed” or “differential expression” as used herein refers to a difference in the level of expression of the nucleic acids that can be assayed by measuring the level of expression of the products of the nucleic acids, such as the difference in level of messenger RNA transcript or a portion thereof expression or of proteins expressed of the nucleic acids.

The term “difference in the level of expression” refers to an increase or decrease in the measurable expression levels of a given nucleic acid, for example as measured by the amount of messenger RNA transcript and/or the amount of protein in a test sample as compared with the measureable expression level of a given nucleic acid in a reference sample (i.e. control subject with indolent OSCC). In one embodiment, the differential expression can be compared using the ratio of the level of expression of a given nucleic acid or nucleic acids as compared with the expression level of the given nucleic acid or nucleic acids of a reference sample (i.e a control subject with indolent OSCC), wherein the ratio is not equal to 1.0. For example, an RNA or protein is differentially expressed if the ratio of the level of expression of a first sample as compared with a second sample is greater than or less than 1.0. For example, a ratio of greater than 1, 1.2, 1.5, 1.7, 2, 3, 3, 5, 10, 15, 20 or more, or a ratio less than 1, 0.8, 0.6, 0.4, 0.2, 0.1, 0.05, 0.001 or less. In another embodiment, the differential expression is measured using p-value. For instance, when using p-value, a nucleic acid is identified as being differentially expressed between a first sample and a second sample when the p-value is less than 0.1, preferably less than 0.05, more preferably less than 0.01, even more preferably less than 0.005, the most preferably less than 0.001.

Depending on the sample used for reference expression levels, the difference in the level of expression may or may not be statistically significant. For example, if the sample used for reference expression levels is from a subject or subjects with aggressive HNSCC, then when the difference in the level of expression is not significantly different, the subject may have aggressive HNSCC. However, when the difference in the level of expression is significantly different, the subject may have indolent SCCHN.

In a preferred embodiment, the difference is statistically significant. For example, if the sample used for reference expression levels is from a subject or subjects with indolent HNSCC, then when the difference in the level of expression is not significantly different, the subject may have indolent HNSCC. However, when the difference in the level of expression is significantly different, the subject may have aggressive SCCHN.

The term “test sample” as used herein refers to any fluid, cell or tissue sample from a subject which may be assayed for nucleic acid expression products and/or reference expression levels, including for example an isolated RNA fraction, optionally mRNA for nucleic acid determinations. The test sample may be tissue taken by biopsy (e.g. prior to surgical resection) or during surgical resection or following surgical resection. The test sample may from the mouth, head or neck, for example tissue from the oral cavity such as buccal, floor of the mouth (FOM), tongue, alveolar, retromolar, palate, gingival, or other oral tissue, the pharynx, the larynx, the paranasal sinuses and nasal cavity or the salivary glands. The sample for example may comprise formalin fixed and/or paraffin embedded tissue, a frozen tissue or fresh tissue. The sample may be used directly as obtained from the source or following a pretreatment to modify the character of the sample, e.g. to obtain a RNA or polypeptide fraction. Where the control is RNA, the control RNA may also be referred to as reference RNA. Reference RNA may include for example a universal RNA pool.

The term “subject” as used herein refers to any member of the animal kingdom that is capable of having HNSCC. Suitable subjects include, but are not limited to, a human, a livestock animal, a companion animal, a lab animal, and a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In yet another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In specific embodiments, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc. In an exemplary embodiment, a subject is human. Specifically, a subject may be a human being that has OSCC or that is suspected of having OSCC.

The present invention may also be used to determine the aggressiveness of head and neck squamous cell carcinoma (HNSCC). The term “head and neck squamous cell carcinoma” or “HNSCC” as used herein refers to cancers of the squamous cells that line the mucosal surfaces of the head and neck. HNSCC may be categorized by area of the head and neck and includes the oral cavity, the pharynx (nasopharynx, oropharynx, and hypopharynx), the larynx, the paranasal sinuses and nasal cavity and the salivary glands. In a specific embodiment, the present invention may also be used to determine the aggressiveness of oral squamous cell carcinoma (OSCC). The term “oral squamous cell carcinoma” or “OSCC” as used herein refers to a subtype of head and neck cancers that includes squamous cell carcinomas of the oral cavity. The squamous cell carcinomas of the oral cavity can affect, for example, buccal, floor of the mouth (FOM), tongue, alveolar, retromolar, palate, gingival, or other oral tissue. All stages and metastasis are included.

The term “indolent” as used herein refers to a tumor or cells that grow slowly and/or do not metastasize. An indolent tumor may be a pathologically organ-confined cancer. An indolent tumor is associated with low morbidity and favorable outcomes. Histopathologic criteria such as perineural or lymphovascular invasion and tumor depth are predictors of indolence. In the present invention, the nucleic acid molecular signature is differentially expressed in aggressive tumors relative to indolent tumors.

The term “risk” as used herein refers to the probability that an event will occur over a specific time period, for example, as in the metastasis of HNSCC within 12, 18, or 24 months after surgery, in a subject diagnosed and surgically treated for HNSCC and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1-p) where p is the probability of event and (1-p) is the probability of no event) to no-conversion.

The molecular signature described herein may be used to select treatment for HNSCC patients. As explained herein, the nucleic acids can classify HNSCC as aggressive and into groups that might benefit from aggressive therapy. In an embodiment, a subject classified as having aggressive HNSCC may be treated. In another embodiment, a subject indicated to have a poor prognosis may be treated. In still another embodiment, a subject indicated to have a poor prognosis may be more aggressively treated. A skilled artisan would be able to determine standard treatment versus aggressive treatment. Accordingly, the methods disclosed herein may be used to select treatment for HNSCC patients. In an embodiment, the subject is treated based on the difference in expression relative to the reference expression level. This classification may be used to identify groups that are in need of treatment or not or in need of more aggressive treatment. The term “treatment” or “therapy” as used herein means any treatment suitable for the treatment of HNSCC. For example, HNSCC may be treated with surgery, radiation and/or adjuvant chemotherapy. The term “adjuvant chemotherapy” as used herein means treatment of cancer with chemotherapeutic agents after surgery where all detectable disease has been removed, but where there still remains a risk of small amounts of remaining cancer. Non-limiting examples of chemotherapeutic agents include cisplatin, carboplatin, vinorelbine, gemcitabine, doccetaxel, paclitaxel and navelbine. In some embodiments, the treatment is chemotherapy. In other embodiments, the treatment is radiotherapy.

### II. Kit

According to a further aspect, there is provided a kit to determine the aggressiveness of HNSCC in a subject, comprising detection agents that can detect the expression products of at least 10 nucleic acids selected from Table A, and instructions for use. Additionally, it is realized that the kit may also comprise detection agents that can detect the expression products of at least one or more of the nucleic acids described herein in Table B. The kit may further comprise one or more nucleic acids used as a normalization control. The kit may comprise detection agents that can detect the expression products of 10 to 20, 20 to 30, 30 to 50, 50 to 100, 100 to 200, 200 to 300, 300 to 400 and more than 400 nucleic acids described herein.

According to a further aspect, there is provided a kit to select a therapy for a subject with HNSCC, comprising detection agents that can detect the expression products of at least 10 nucleic acids selected from Table A, and instructions for use. Additionally, it is realized that the kit may also comprise detection agents that can detect the expression products of at least one or more of the nucleic acids described herein in Table B. The kit may further comprise one or more nucleic acids used as a normalization control. The kit may comprise detection agents that can detect the expression products of 10 to 20, 20 to 30, 30 to 50, 50 to 100, 100 to 200, 200 to 300, 300 to 400 and more than 400 nucleic acids described herein.

A person skilled in the art will appreciate that a number of detection agents can be used to determine the expression of the nucleic acids. For example, to detect RNA products of the biomarkers, probes, primers, complementary nucleotide sequences or nucleotide sequences that hybridize to the RNA products can be used.

Accordingly, in one embodiment, the detection agents are probes that hybridize to the at least 10 nucleic acids in the molecular signature. A person skilled in the art will appreciate that the detection agents can be labeled. The label is preferably capable of producing, either directly or indirectly, a detectable signal. For example, the label may be radio-opaque or a radioisotope, such as ³H, ¹⁴C, ³²P, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion.

The kit can also include a control or reference standard and/or instructions for use thereof. In addition, the kit can include ancillary agents such as vessels for storing or transporting the detection agents and/or buffers or stabilizers.

As various changes could be made in the above compounds, products and methods without departing from the scope of the invention, it is intended that all matter contained in the above description and in the examples given below, shall be interpreted as illustrative and not in a limiting sense.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Introduction to Examples 1-8

Aggressive carcinogen-induced oral squamous cell carcinomas (OSCC) are difficult to treat due to locoregional recurrences. In contrast, more indolent lesions can be treated with single modality surgical intervention with low morbidity and favorable outcomes. Histologic criteria such as perineural or lymphovascular invasion and tumor depth, harbingers of early spread to regional lymph nodes, are commonly used to predict tumor behavior^{1,2}. Additionally, among clinical staging criteria, metastatic lymphadenopathy is one of the best predictors of a poor prognosis as it likely reflects aggressive primary tumor biology³⁻⁵ (seer.cancer.gov/statfacts/html/oralcay.html). This staging is especially challenging in early disease as 20% of these patients have pathologically identifiable disease that is clinically undetectable. Thus, all “high risk” patients undergo neck dissection operations, which prove to be unnecessary in nearly 80% of clinically node negative patients. However, there is a dearth of studies delineating markers predictive of lymph node involvement, and genetic stratification approaches are at an early stage^{6,7}. In addition, the molecular underpinnings of aggressive OSCC growth and metastasis remain largely undefined^{5,8}.

Next generation sequencing (NGS) of human head and neck squamous cell carcinomas (HNSCC), of which OSCC are a significant subset, has confirmed previously identified aberrations (e.g. TP53 and CDKN2A) and has also defined novel NOTCH and FAT gene mutations along with frequent P13K pathway mutations⁹⁻¹⁴. In addition, other mitogenic cascades, such as RAS and JAK/STAT, are altered at lower frequencies. In contrast, mutations that distinguish indolent from aggressive human OSCC remain undefined. Genomic approaches to identify signatures that predict metastatic behavior in OSCC have been described but none have approached the clinical impact of tests available for breast cancer and ocular melanoma¹⁵⁻¹⁹. Importantly, molecular clues reflecting metastatic regulators have not arisen from these biomarker studies.

To better understand the genomic basis of the aggressive OSCC phenotype, the inventors employed their recently described carcinogen-induced mouse oral cancer (MOC) cell line model²⁰. These MOC lines, which parallel the distinct phenotypes seen in human disease, are either CD44I^{ow} and indolent, or CD44^{high} and aggressive/metastatic. Herein, the inventors used genomic approaches to (1) define parallels to human OSCC, (2) understand the transcriptomic differences that underlie both phenotypes and (3) translate this information into a clinically relevant context. Remarkably, despite differences in species and carcinogen exposure, many of the same drivers implicated in humans were altered in MOC lines, revealing highly conserved pathways in OSCC tumorigenesis. In addition, the inventors identified a gene expression signature associated with metastasis that was conserved from mouse to three distinct human datasets, uncovering potential promoters of aggressive OSCC. Finally, the inventors successfully translated this signature into a platform for potential clinical application. Together, this analysis identifies novel pathways associated with aggressive growth and metastasis that may contribute functionally to cancer progression and lead to improved diagnostics.

### Example 1. Next Generation Sequencing to Determine Somatic Alterations Between Mouse Oral Carcinoma Cell Lines

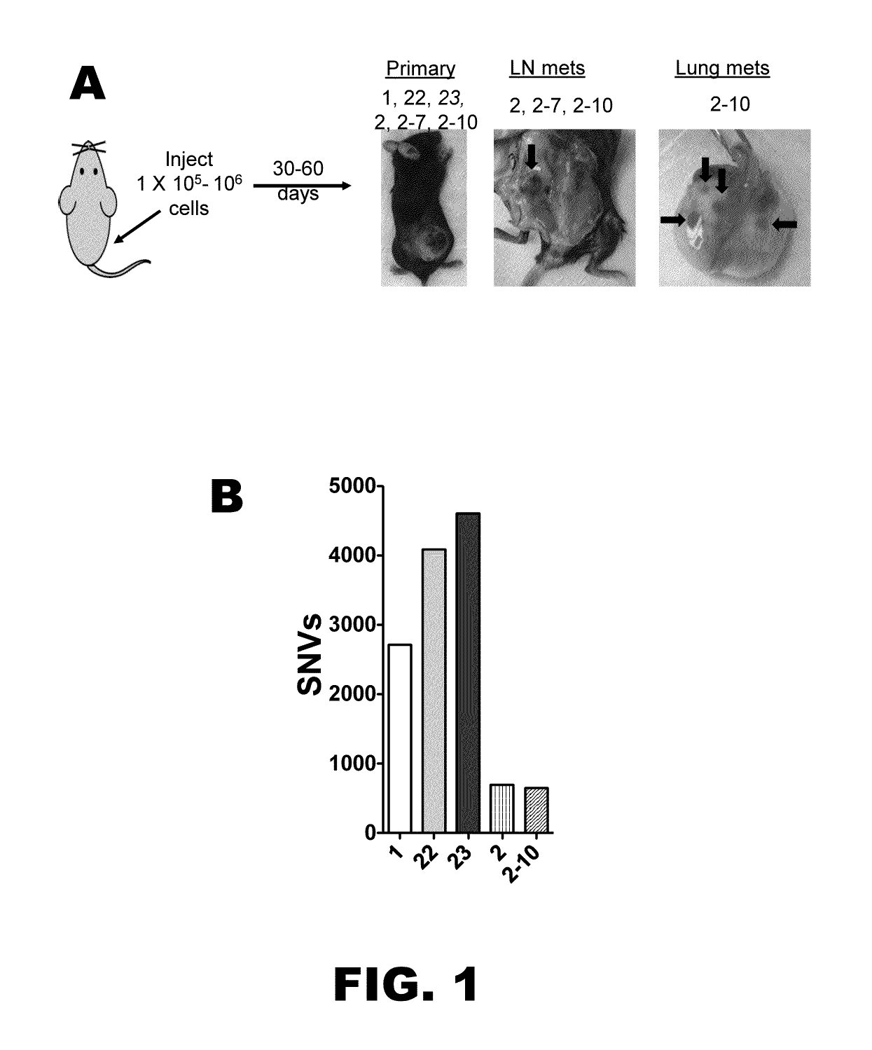

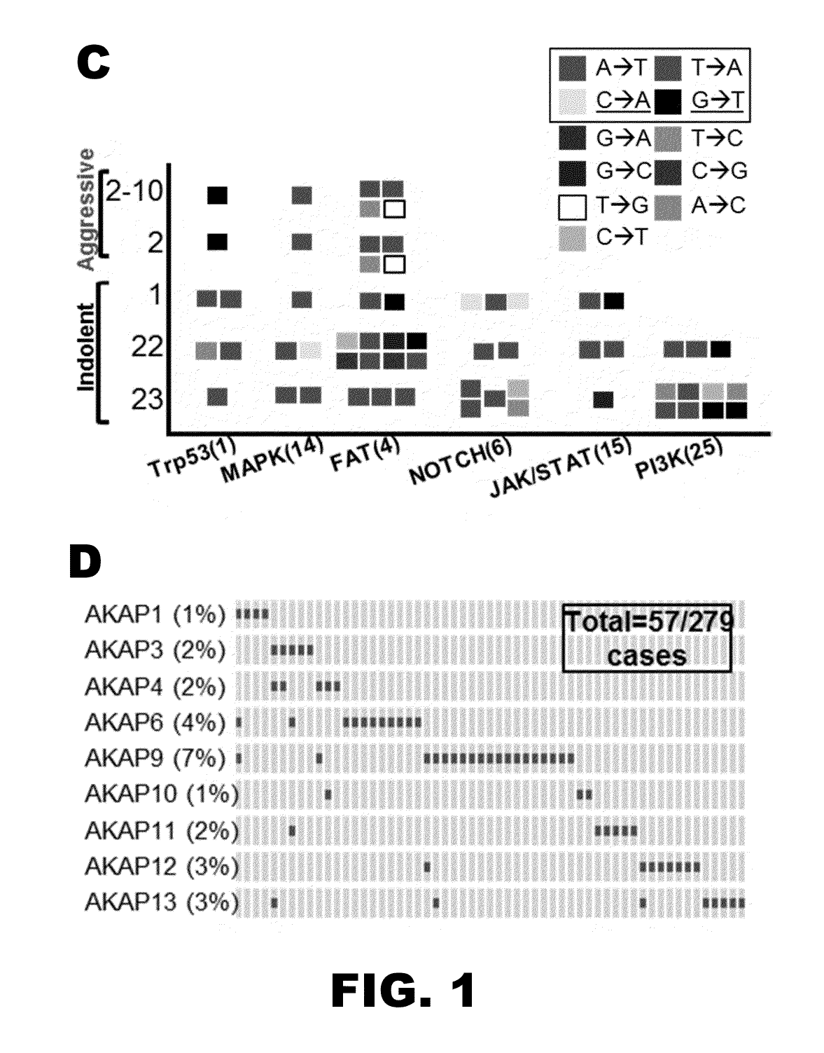

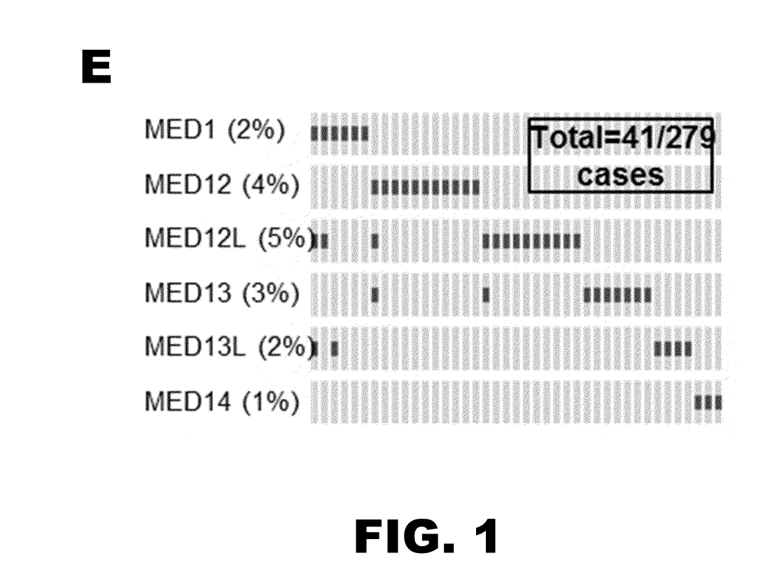

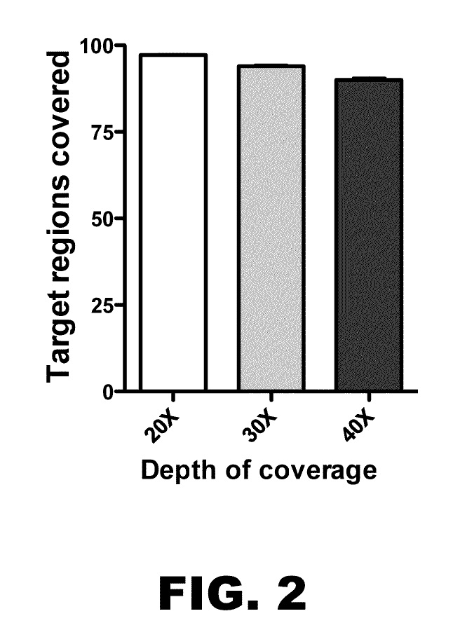

Previously, the inventors described a 7,12 dimethylbenzanthracene (DMBA)-induced mouse cell line model of OSCC where, upon transplantation, individual lines displayed fixed in vivo phenotypes (FIG. 1A). The indolent lines all formed tumors in RAG2^{-/-} immunodeficient mice but only MOC1 and MOC22 grew in wild type mice. Of the aggressive lines, MOC2-7 and MOC2-10 were derived from the MOC2 line, but the MOC2-10 line was included in the current analysis because it uniquely displayed lung in addition to lymph node metastasis. Note that the inventors used the flank model for ease of tumor measurement, but lymph node metastasis was also observed upon orthotopic transplantation²⁰. MOC growth behaviors were consistent with human OSCC clinical behavior leading the inventors to investigate whether their somatic alterations were also congruent. NGS was performed on 3 indolent and 2 related aggressive/metastatic lines with excellent coverage depth (FIG. 2).

### Example 2. Overview of Mutations Identified in Mouse Oral Carcinoma Cell Lines

Many somatic non-synonymous single nucleotide variants (nsSNVs) were identified in these lines as expected for carcinogen treated mouse tumors (FIG. 1B, Table 1, Table 2 and ²²). The inventors also have data for all SNVs identified in indolent lines (data not shown). Observed mutations were consistent with the known predilection of DMBA for first, A:T→T:A (range 48.7-59.3% of total) and second, G:C→T:A (range of 14.4-17.6%) transversions (FIG. 1C, Table 3)²³ overlapping with G:C→T:A mutations described for HNSCC¹¹.

### Example 3. Conservation of Candidate Driver Mutations between MOC Lines and Human HNSCC

The inventors next compared the MOC line mutations with the 32 most significantly mutated genes from the TCGA HNSCC effort (Table 4). Surprisingly, as a group, the MOC lines bore mutations in many of these same genes with seven of the top ten genes altered in human HNSCC also carrying mutations in the MOC lines (Table 5). The inventors also asked whether drivers described in human OSCC were present in MOC lines. Recent work has highlighted the NOTCH, P13K, MAPK, JAK/STAT, FAT families and Trp53 as pathways critical for HNSCC ⁹⁻¹³. Again, MOC lines bore mutations in the same driver pathways described for HNSCC and changes were typical of the DMBA spectrum as described above (FIG. 1C, Table 6). Whereas all MOC lines had mutations in Trp53, MAPK and the FAT family of genes, only the indolent cell lines showed NOTCH, JAK/STAT and P13K pathway mutations. Mutations in FAT1 (12%) and FAT4 (10%) have been identified in human HNSCC¹³ and these genes in addition to FAT2 and FATS were altered in MOC lines. Other candidate driver mutations included CASP8 in MOC22, which is altered in 8-10% of human HNSCC typically in association with HRAS mutations^{11,13}. Indels did not segregate into either indolent or aggressive growth categories (Table 7). Copy number and tumor heterogeneity could not be reliably evaluated, as normal tissue from the parental mice was not available. Thus, as a group, MOC lines had alterations in the most commonly mutated genes and driver pathways in HNSCC reflecting an unexpected conservation in the mutational landscape, despite differences in the species, specific carcinogen used to derive the lines and overall numbers of mutations.

### Example 4. Novel Candidate Cancer Genes

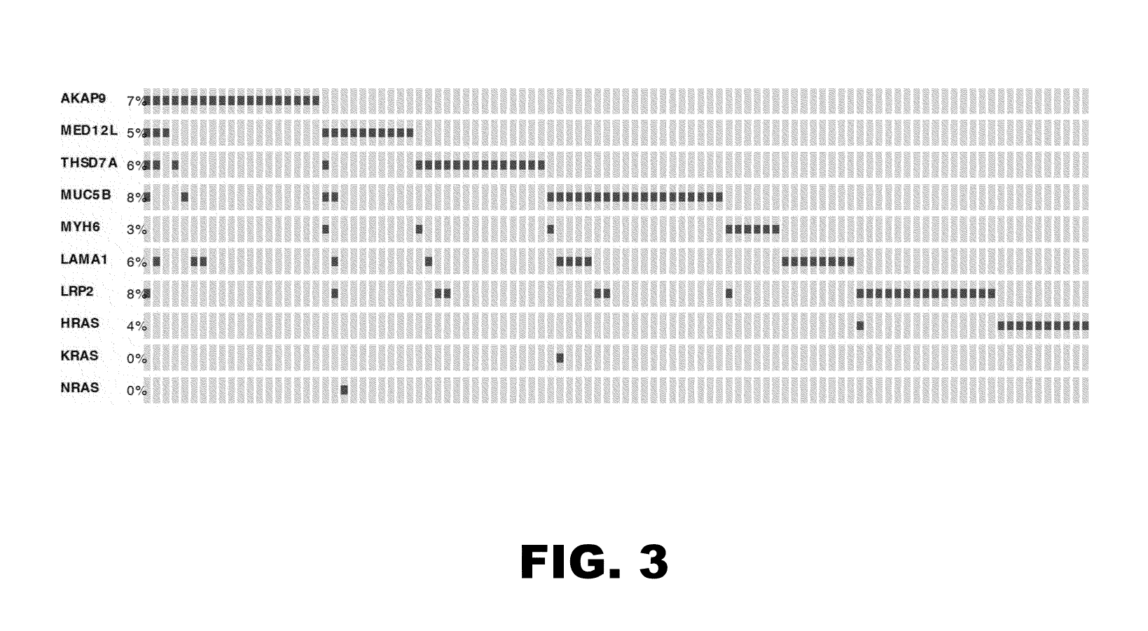

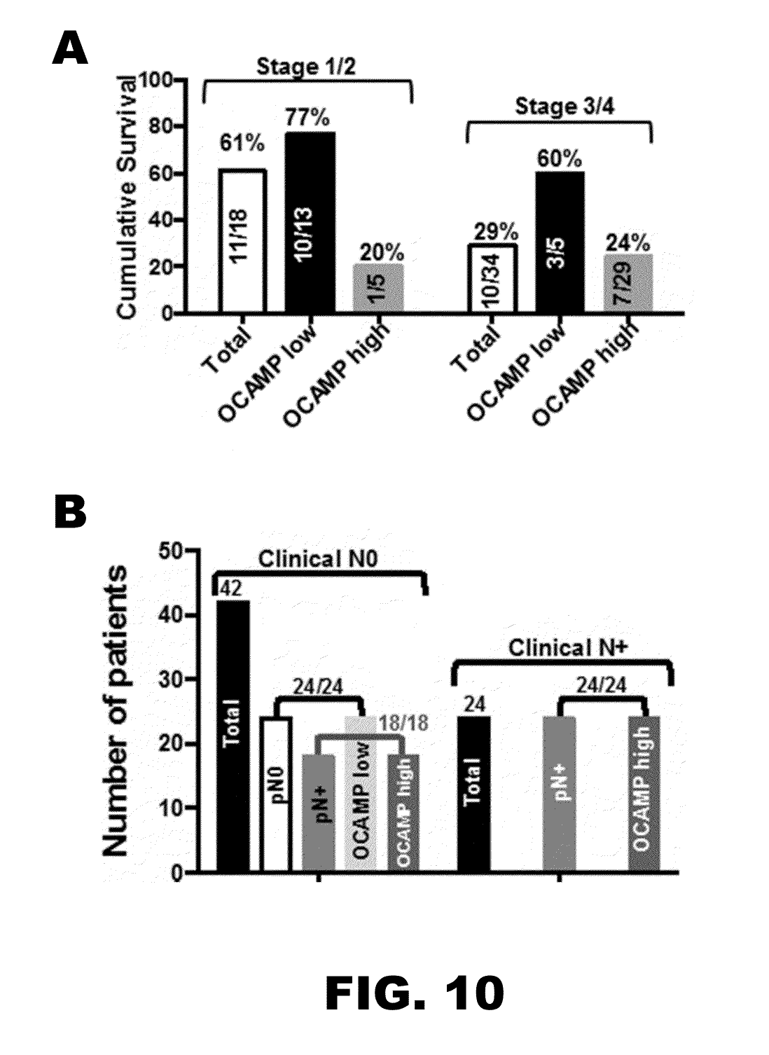

As common MOC line mutations may represent novel OSCC promoters, the inventors' analysis identified the A kinase anchoring protein Akap9, mediator complex component Med12I and Myh6 as potential candidates (Table 5, Table 6, Table 8 and Table 9). AKAP and MED protein families were mutated in the TCGA cohort using the cBio portal ²³ where the inventors found that 9 members of the AKAP family were mutated in 20.4% of tumors, with AKAP9 changes in 7% (FIG. 10). Six components of the mediator complex were mutated in 14.7% of cases, with MED12L changes in 5% (FIG. 1E). Of note, MED1 mutations were previously identified in 5% of HNSCC¹¹. Importantly, MutSigCV analysis did not identify any of these genes as significantly mutated in TCGA when analyzed individually. However, together the mutations in several AKAP family members and mediator components suggest that these pathways may be relevant promoters. Further analysis identified 5% and 9% rates for AKAPs and 13% and 17.5% for MEDs in two independent HNSCC datasets (¹² and ¹¹, respectively). Finally, very recent work using an RNAi in vivo screen identified MYH9 as a putative cancer gene in SCCA and the inventors identified the related MYH6 gene as commonly mutated in MOC lines²⁵. The TCGA dataset shows equivalent mutation rates for both these genes. In addition, THSD7A, MUC5B, MYH6, LRP2 and LAMA1 gene mutations were common in MOC lines (Table 8, Table 9) and were also present in the TCGA cohort (FIG. 3). These alterations illustrate not only the conservation of structural parallels between mouse and human OSCC but also the ability of the mouse model to highlight novel tumor promoters.

### Example 5. Growth Phenotype Specific Mutations

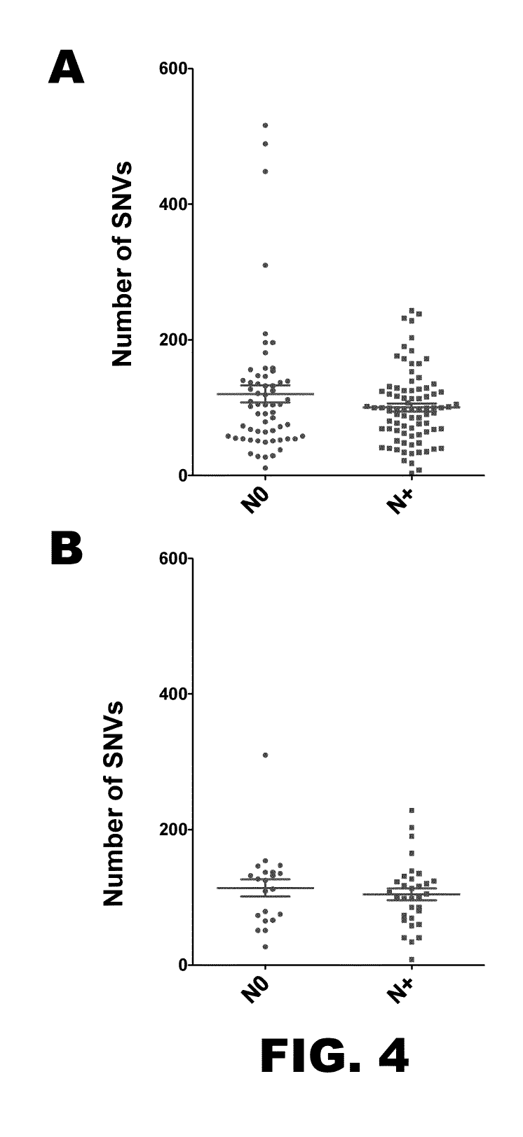

Although NGS confirmed MOC and human OSCC conservation, analysis of mutations specific to indolent or aggressive lines and lymph node versus lung metastatic lines was inconclusive likely due in part to the limited numbers of samples (Table 8, Table 10). The inventors next approached this question by comparing their mouse sequencing data to mutations unique to lymph node metastasis negative (N0, 62 patients) versus positive (N_{+,} 84 patients) OSCC samples from TCGA. The inventors identified 3273 N0 and 3097 N₊ mutations exclusive to each nodal status subset (Table 11). There was no significant difference in the average number of mutations between N0 and N₊ patients regardless of smoking status (FIG. 4A,B and Table 12). This analysis showed 17 nucleic acids commonly mutated in mouse indolent and human NO tumors and 55 common nucleic acids mutated in mouse aggressive and human N₊tumors (Table 13). However, none of these common nucleic acids were mutated at high frequency in the human N0 or N₊ datasets (Table 14, Table 15). Finally, isolated analysis of the human TCGA data also showed that nodal status specific mutations occur infrequently in both the metastatic and non-metastatic tumors (from cBio portal, data not shown). Together, these data suggest that the aggressive OSCC phenotype is not clearly a result of somatic exome changes but rather may be driven instead by epigenetic or transcriptional alterations.

### Example 6. Microarray Analysis Identifies Promoters of Aggressiveness

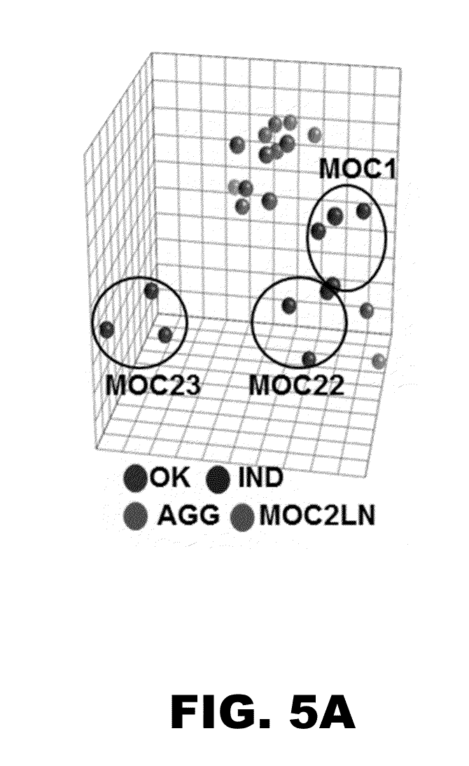

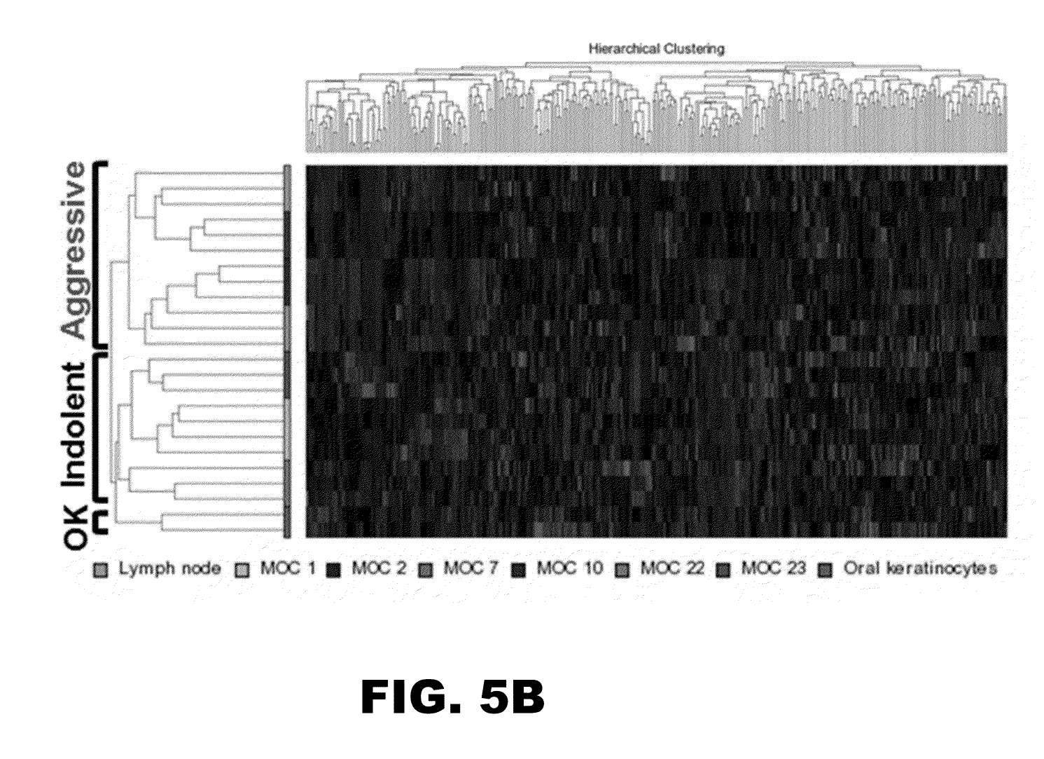

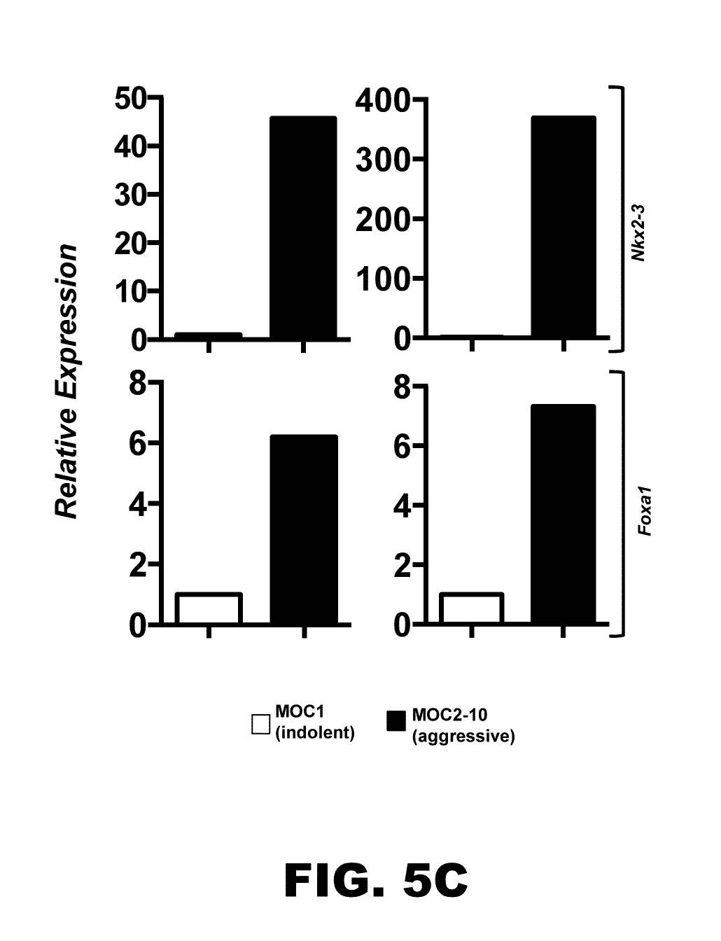

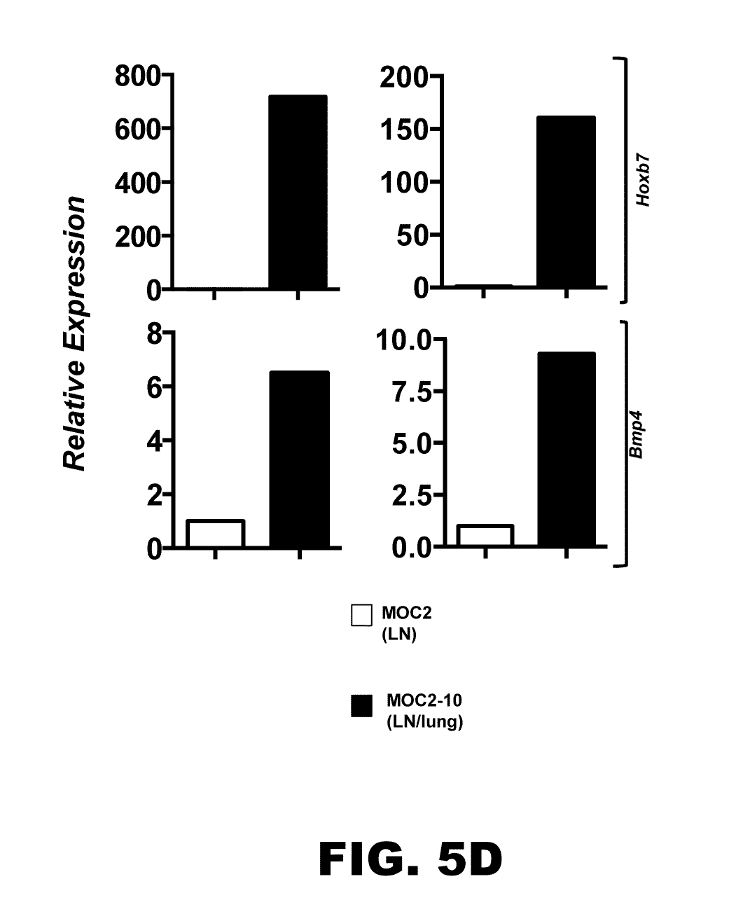

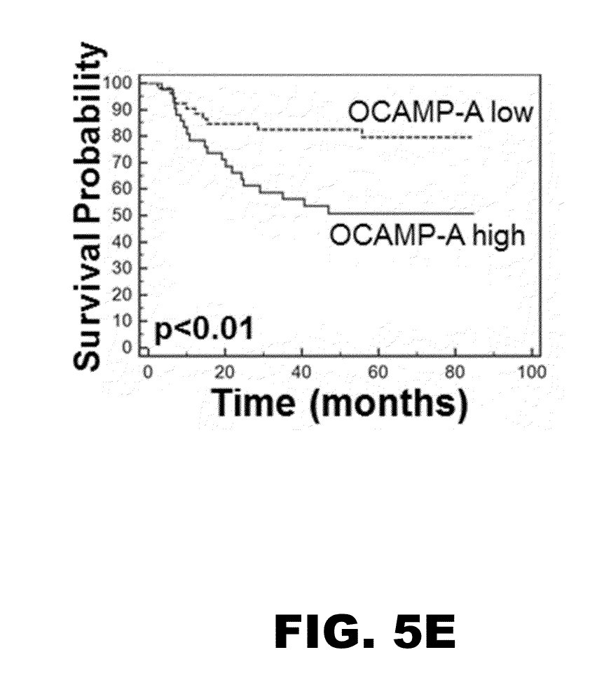

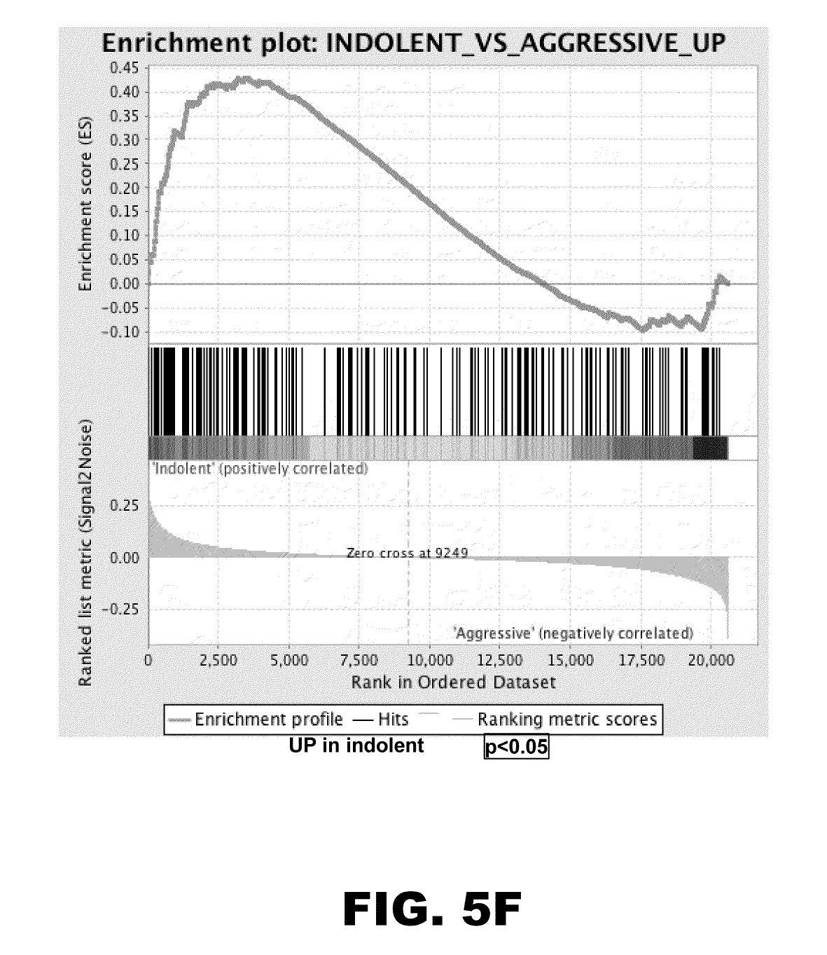

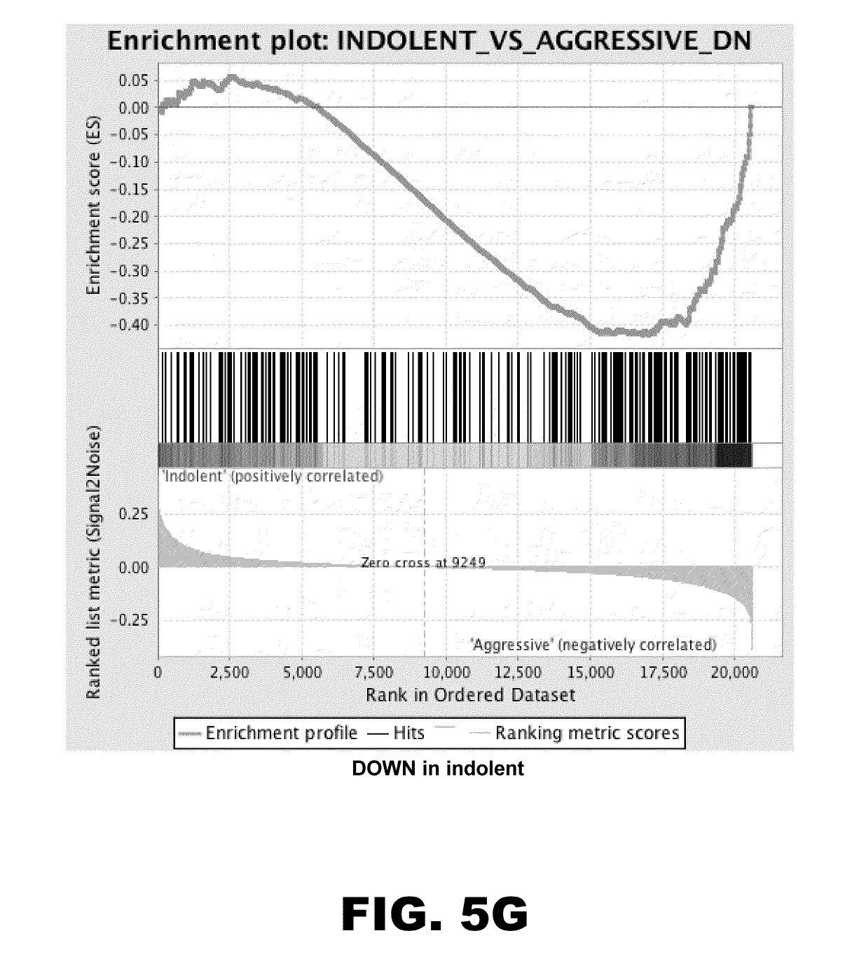

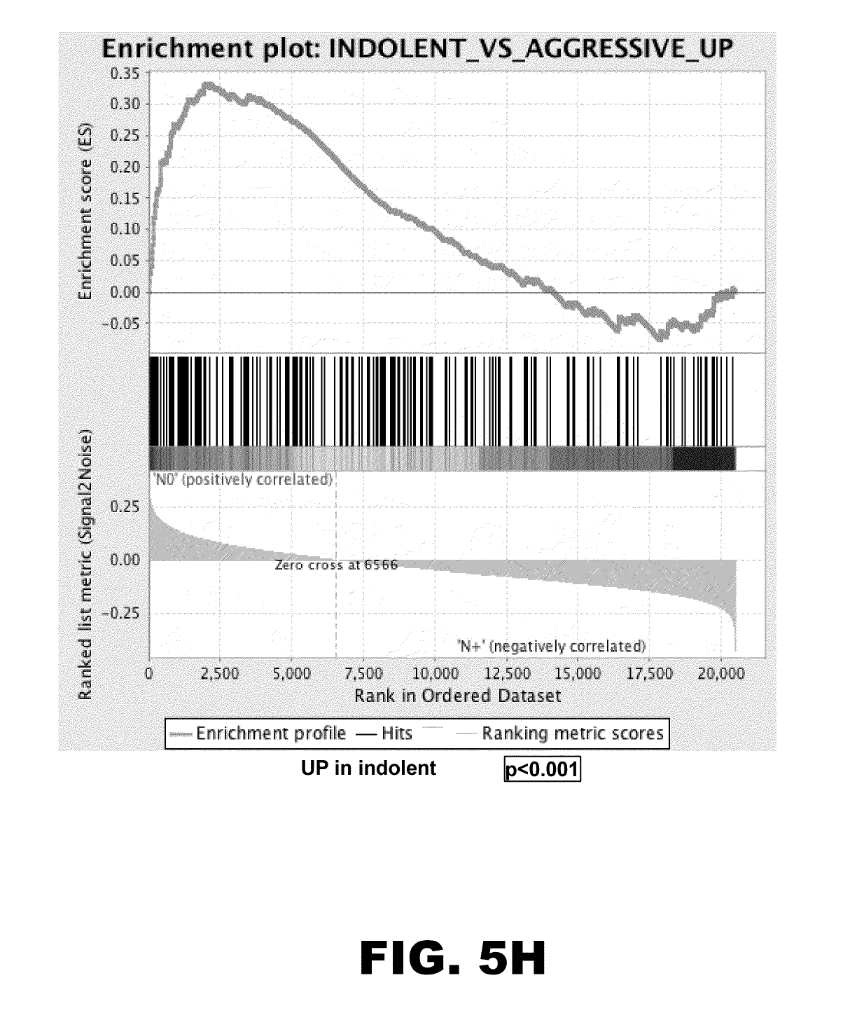

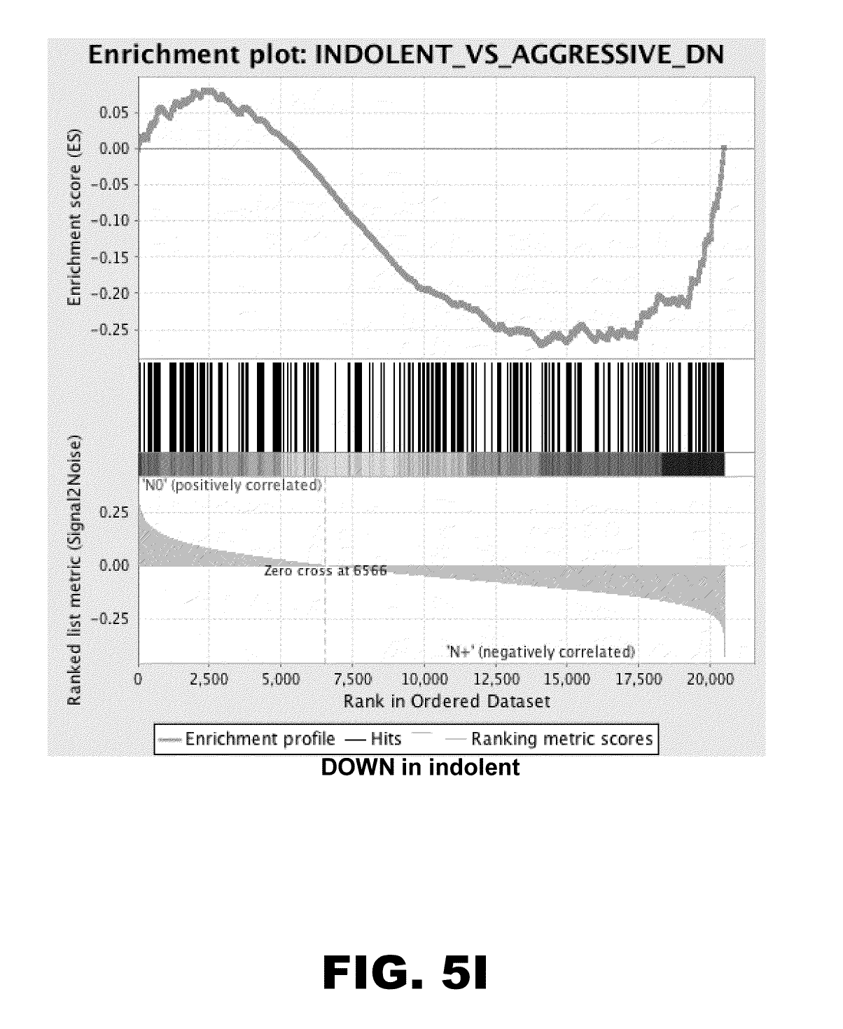

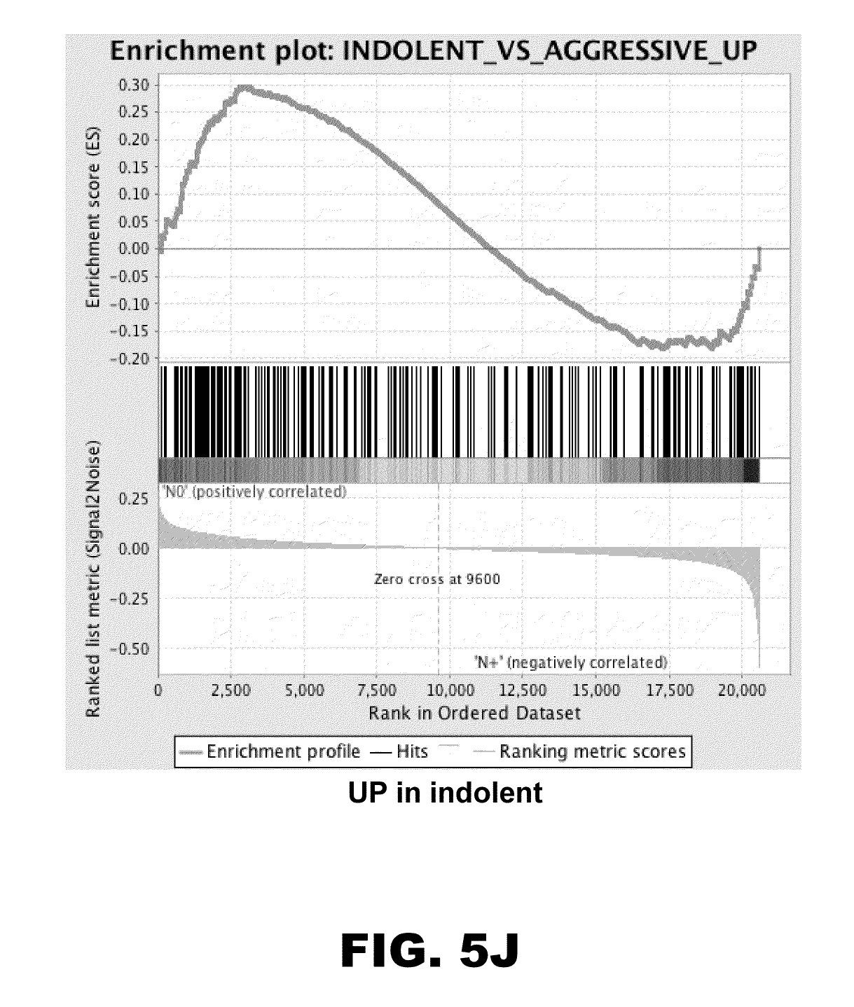

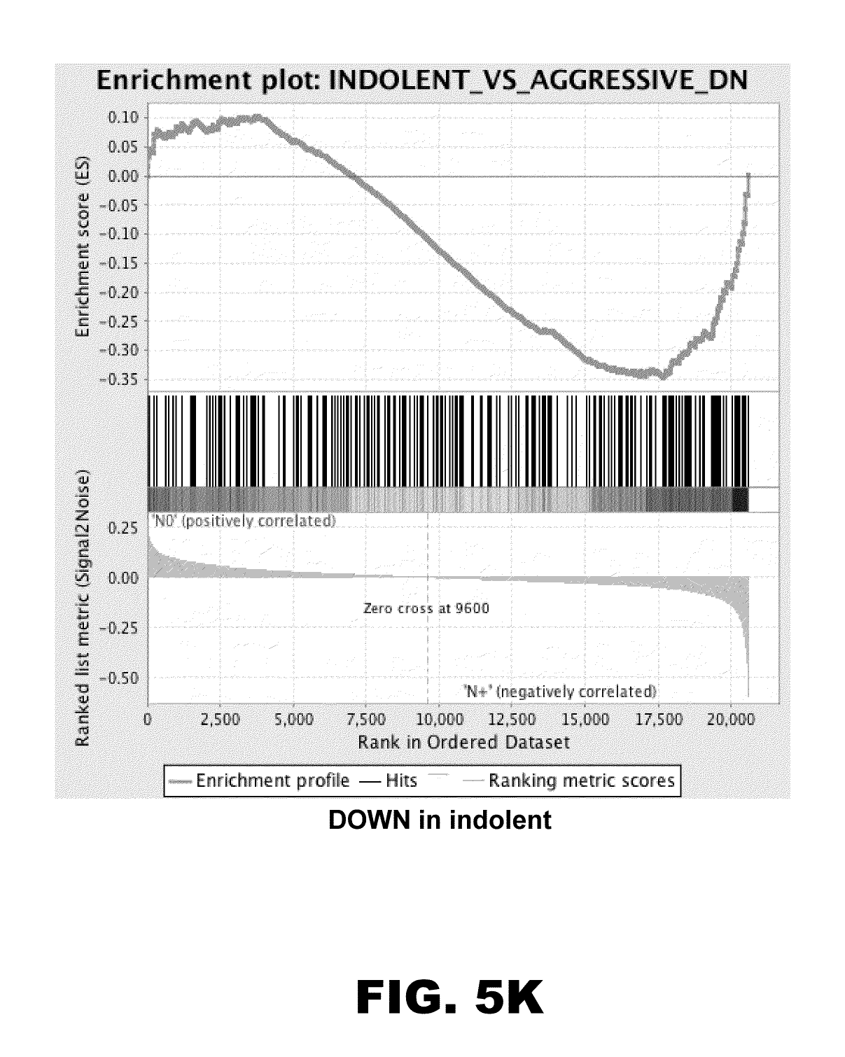

Next, the inventors interrogated MOC lines and primary C57BL/6 oral keratinocytes to identify transcriptomic promoters of aggressiveness. As expected, principal component analysis (PCA) showed that all the related aggressive lines clustered together (FIG. 5A). The indolent MOC1 and MOC22 clustered near each other and were only slightly separated from normal oral keratinocytes. In contrast, MOC23 showed a distinct distribution consistent with it being a unique subtype that grows only in RAG2^{-/-} immunodeficient mice (FIG. 5A).

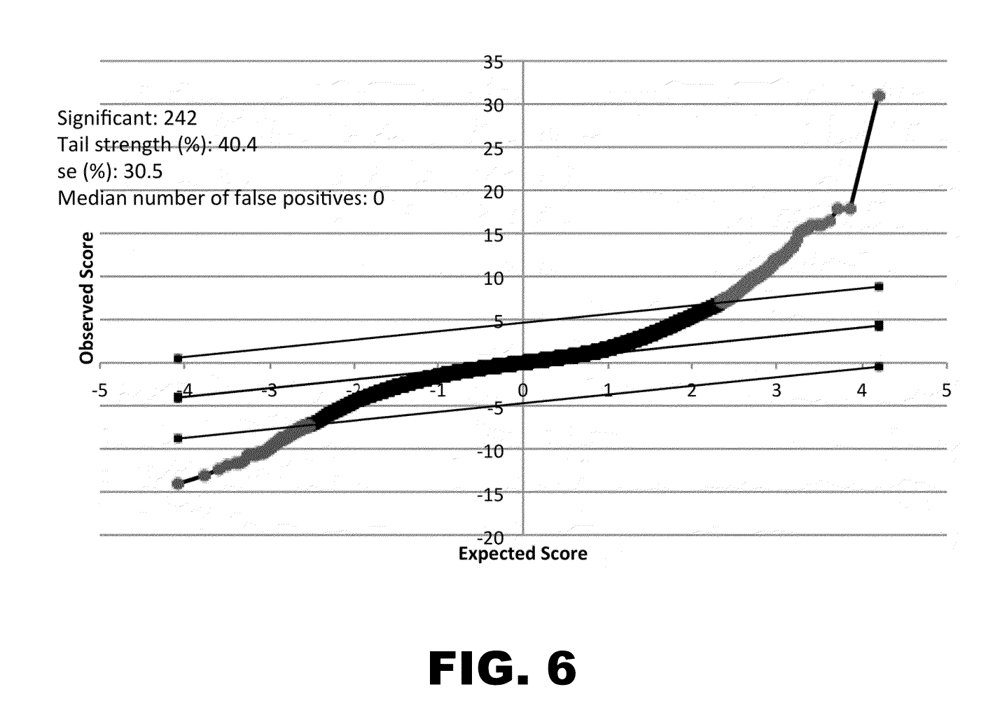

Unsupervised hierarchical clustering demonstrated a metastasis signature (FIG. 5B) and significance analysis of microarrays (SAM) identified specific differentially expressed genes at a false-discovery rate of <10% (FIG. 6, Table 16), which were confirmed by ANOVA (p≦0.01). The mouse signature was divided into significantly downregulated (260) or upregulated (218) genes in indolent versus aggressive lines (Table 17). Expression patters for genes described in human metastatic tumors, such as MUC1, SLPI and TACSTD2, were conserved in mouse OSCC²⁶⁻²⁸. The inventors identified several upregulated transcription factors, including Eomes, Nkx2-3, Foxal, Hnflb, Meisl and E2f4 that were previously not described in OSCC and may be central to controlling global programs of aggressiveness.

The dramatic differences in expression between indolent and aggressive lines for Nkx2-3 and Foxal were confirmed by qRT-PCR (FIG. 5C). Finally, Hoxb7 and Bmp4 were implicated as candidate promoters of lung metastasis as they were overexpressed in MOC2-10 versus MOC2 (FIG. 5D) and are two key candidate promoters of distant metastasis in the MOC model.

### Example 7. Cross Species Aggressiveness/Metastasis Signature Conservation

The inventors next asked whether the mouse signature predicted outcomes in human OSCC patients. Using microarray data from a carcinogen induced, HPV-negative cohort of 97 OSCC patients (UW/FHCRC), the inventors stratified patients based on enrichment of the mouse signature by weighted voting¹⁶. Using Kaplan-Meier analysis, disease specific survival (DSS) was statistically significantly worse for subjects in the group with the more aggressive signature as compared to those with the less aggressive signature (50% versus 80% 5-year DSS, FIG. 5E, p<0.01). Thus, the inventors termed this signature the Oral Cancer Aggressiveness and Metastasis Predictor (OCAMP-A).

To identify overlap between mouse and human signatures, the inventors used Gene Set Enrichment Analysis (GSEA) which allows comparison of data from different platforms and species²¹. Three independent datasets of human OSCC (UW/FHCRC (97 patients), MD Anderson (74 patients²⁹) and TCGA (134 patients) were first classified by stage (WU/FHCRC) or regional lymph node metastasis as surrogate markers of tumor aggressiveness and then independently analyzed by GSEA with OCAMP-A. In all cases there was enrichment of OCAMP-A in human tumors (FIG. 5F-K) that was statistically significant for the TCGA (normalized enrichment score (NES)=1.6, Nominal p-value<0.001) and UW/FHCRC (NES=1.43, Nominal p-value<0.05) but not MD datasets (NES=0.9 and Nominal p-value=0.57).

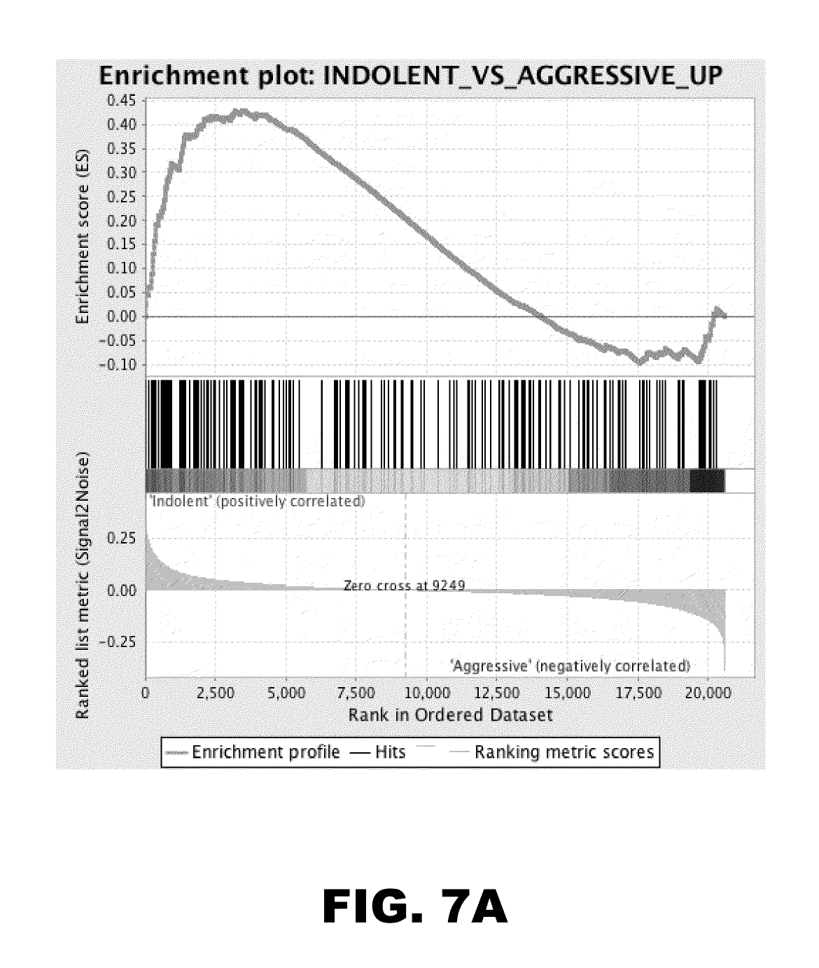

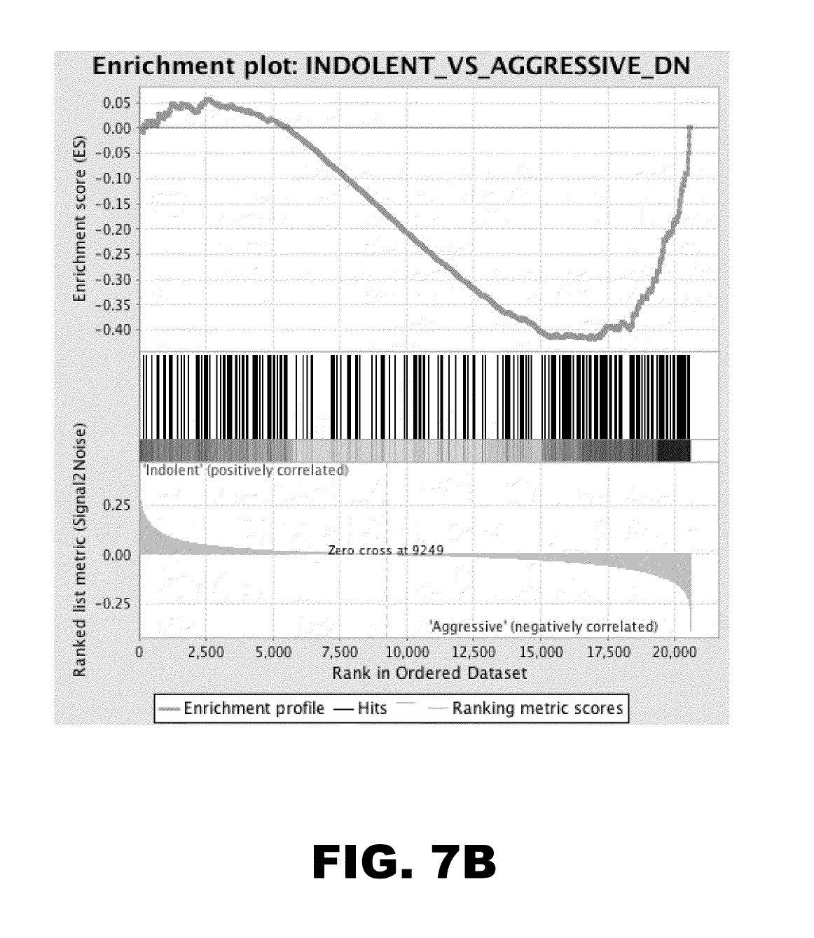

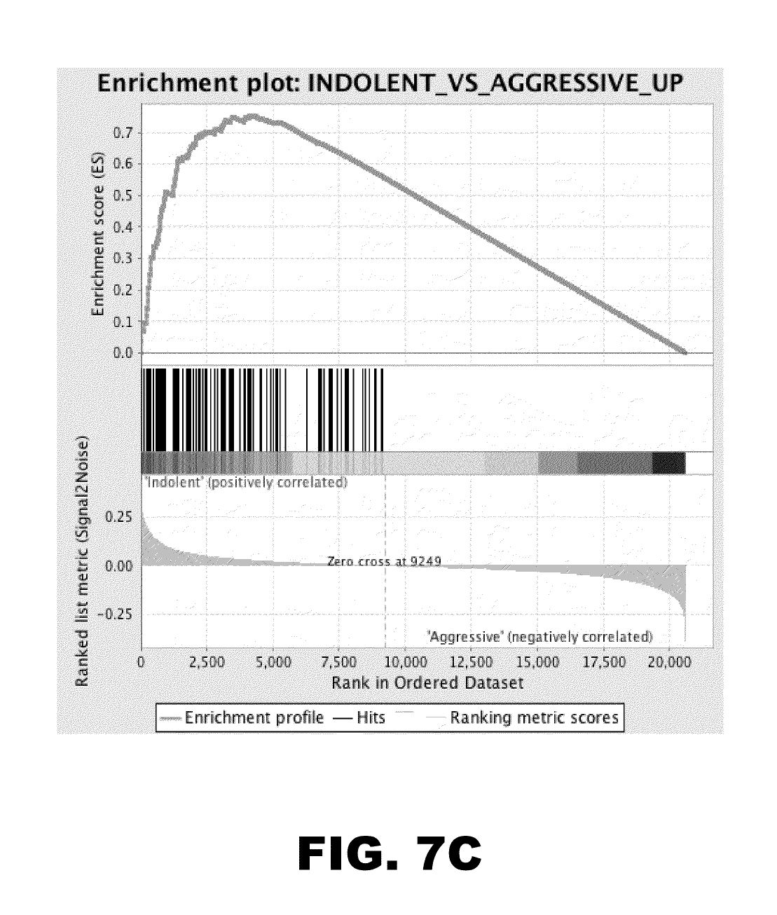

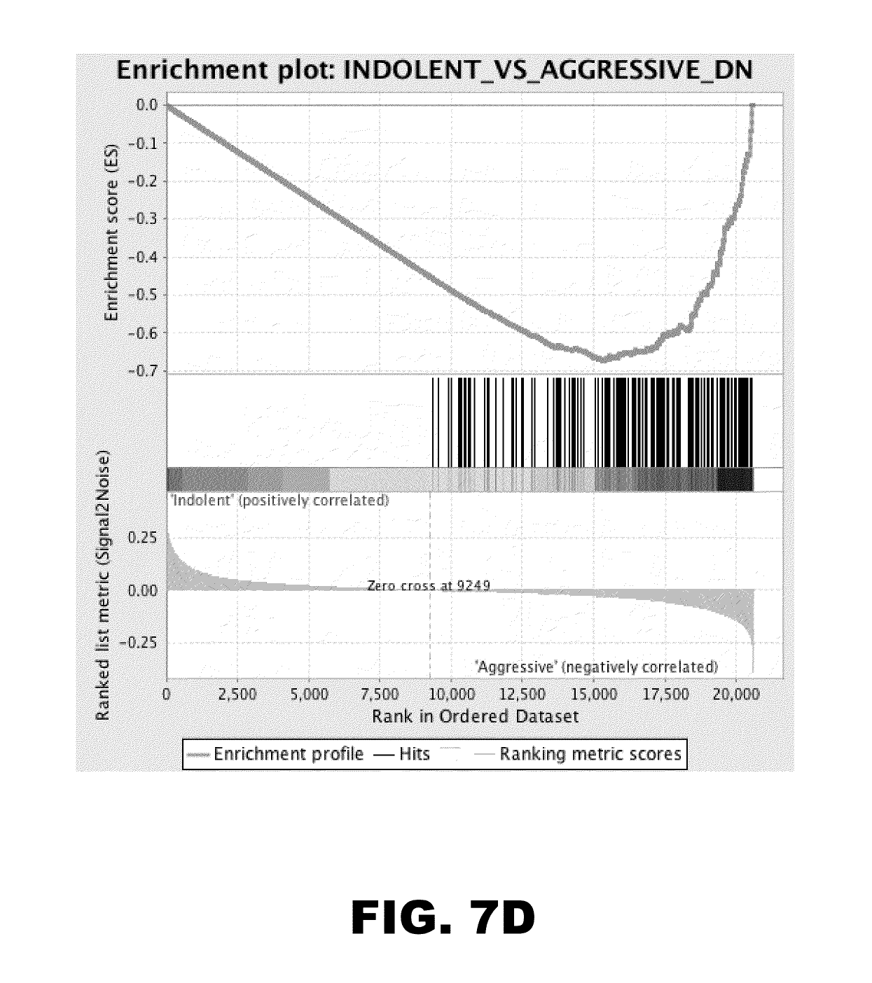

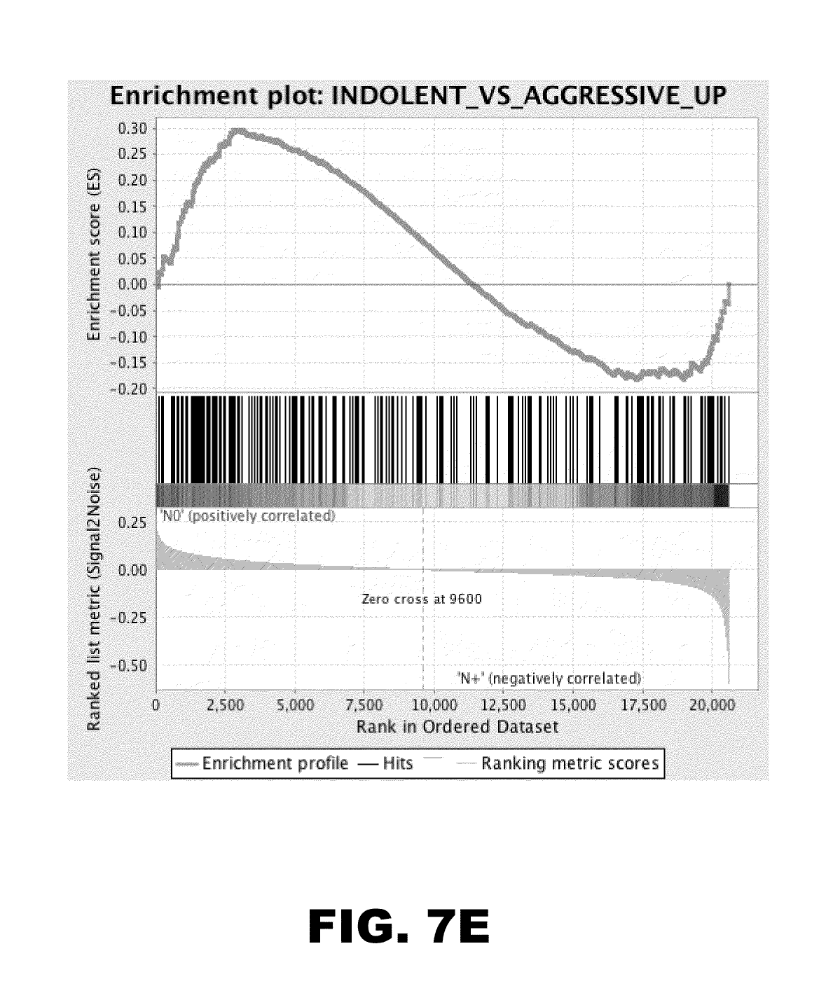

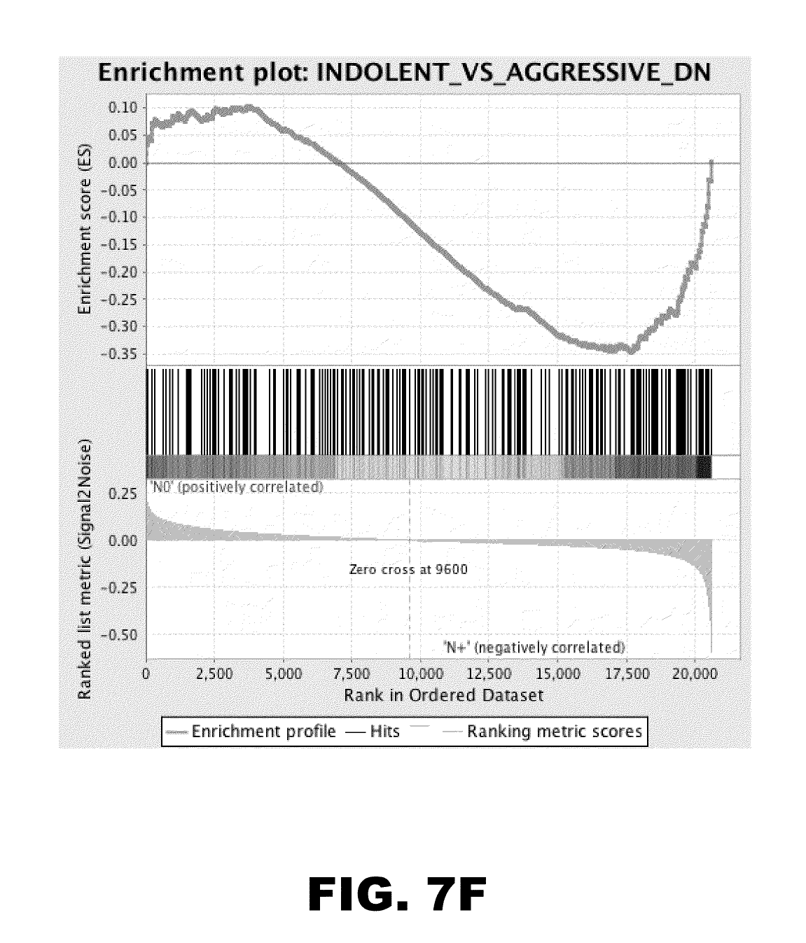

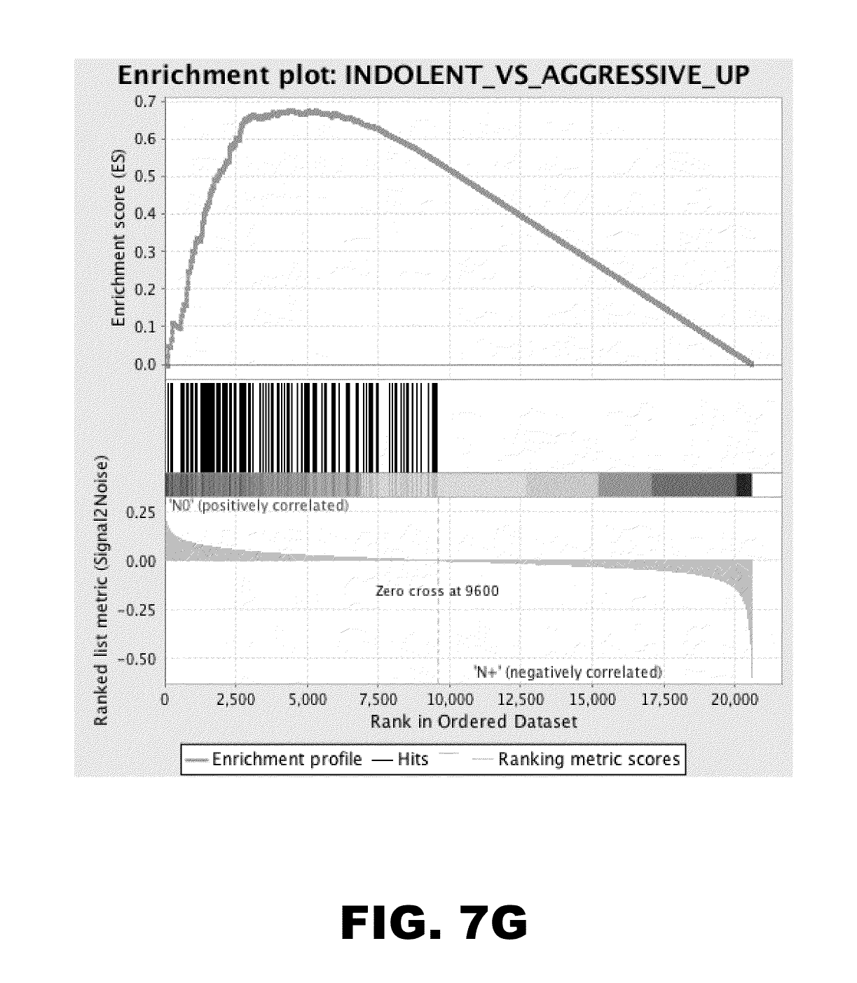

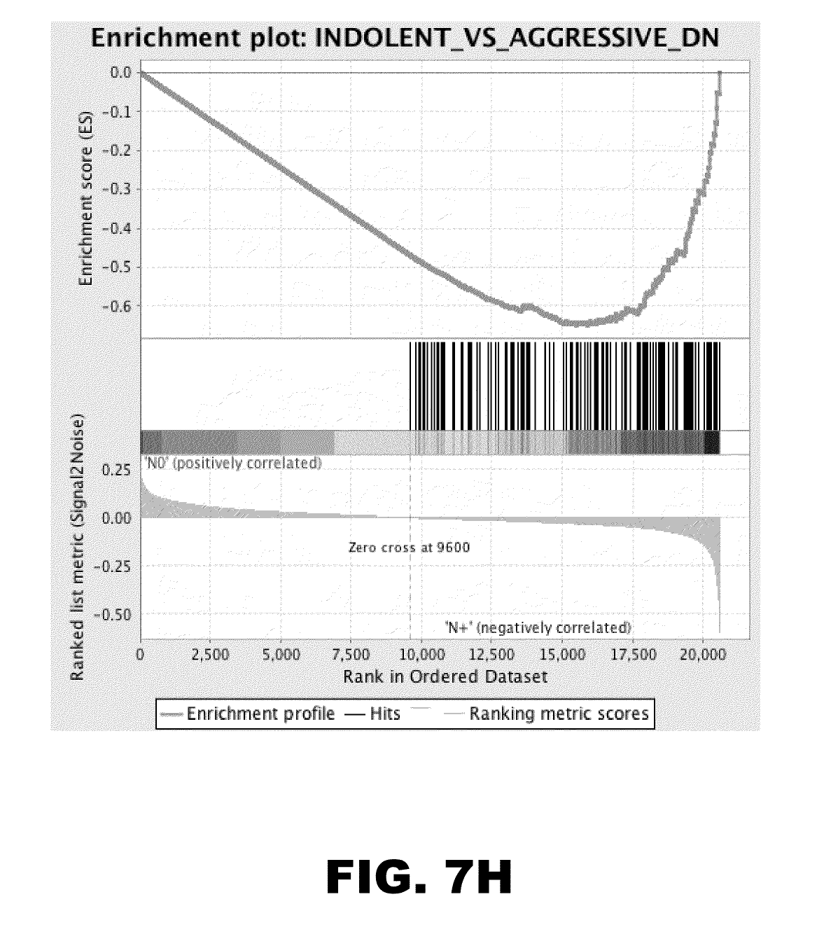

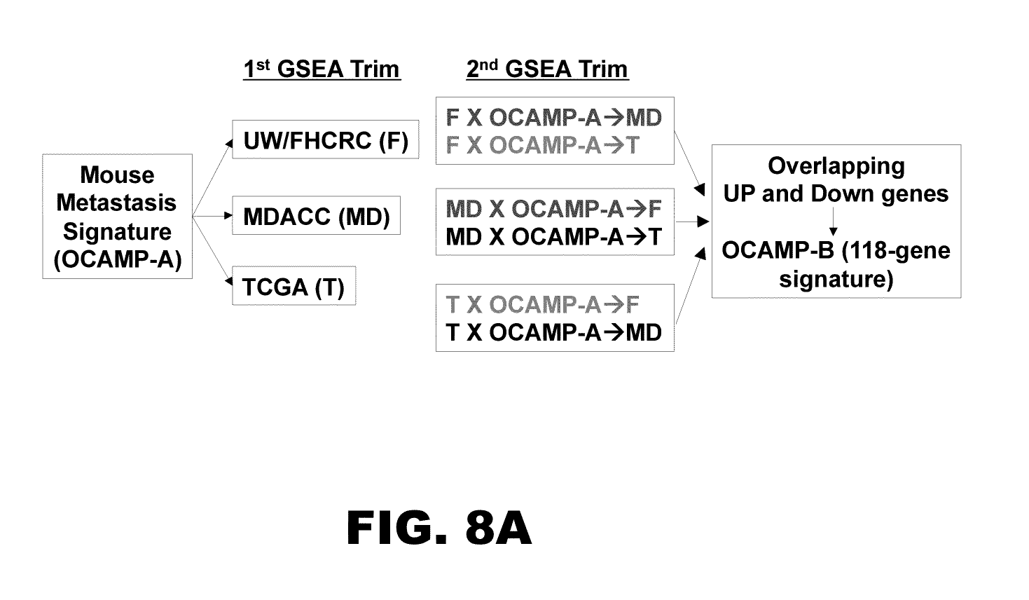

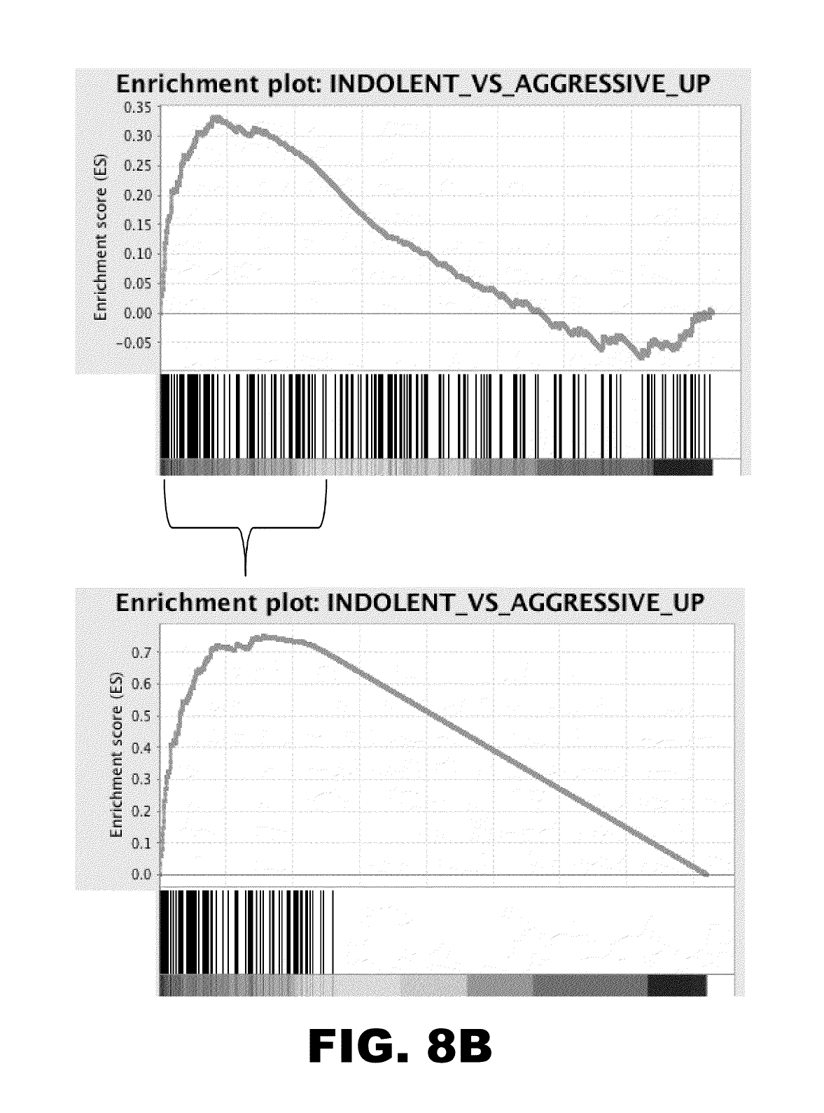

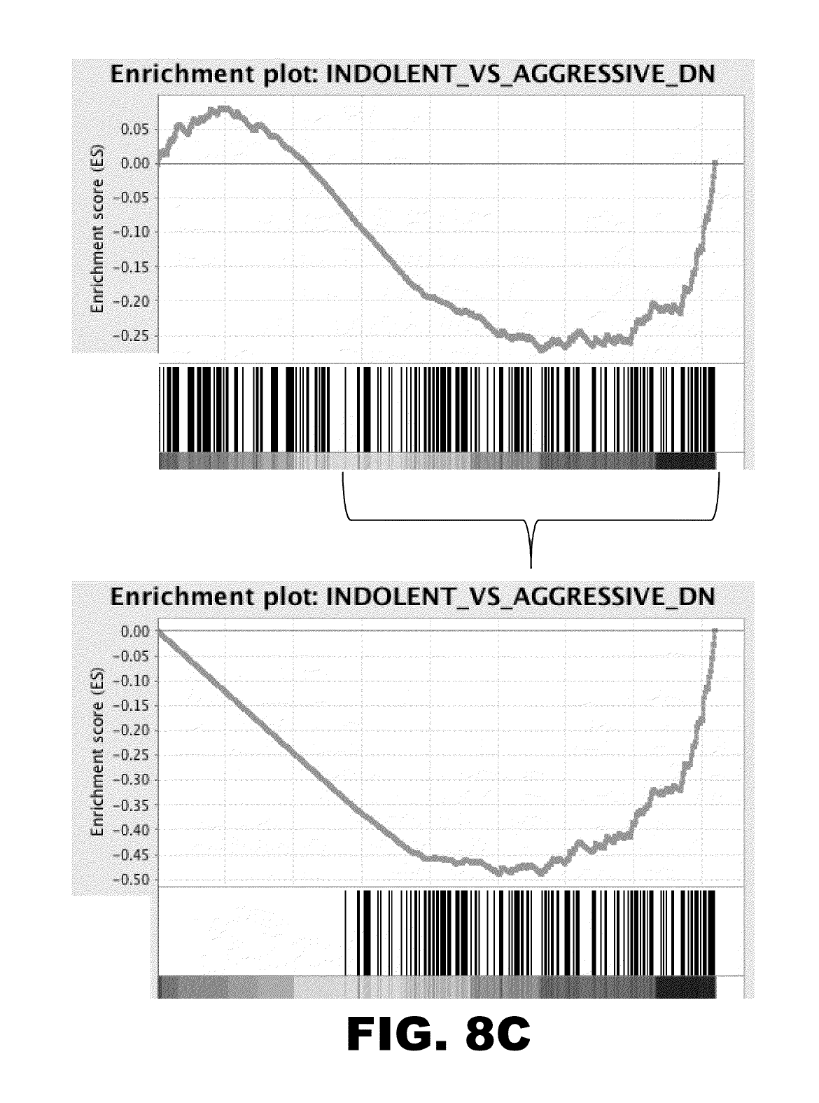

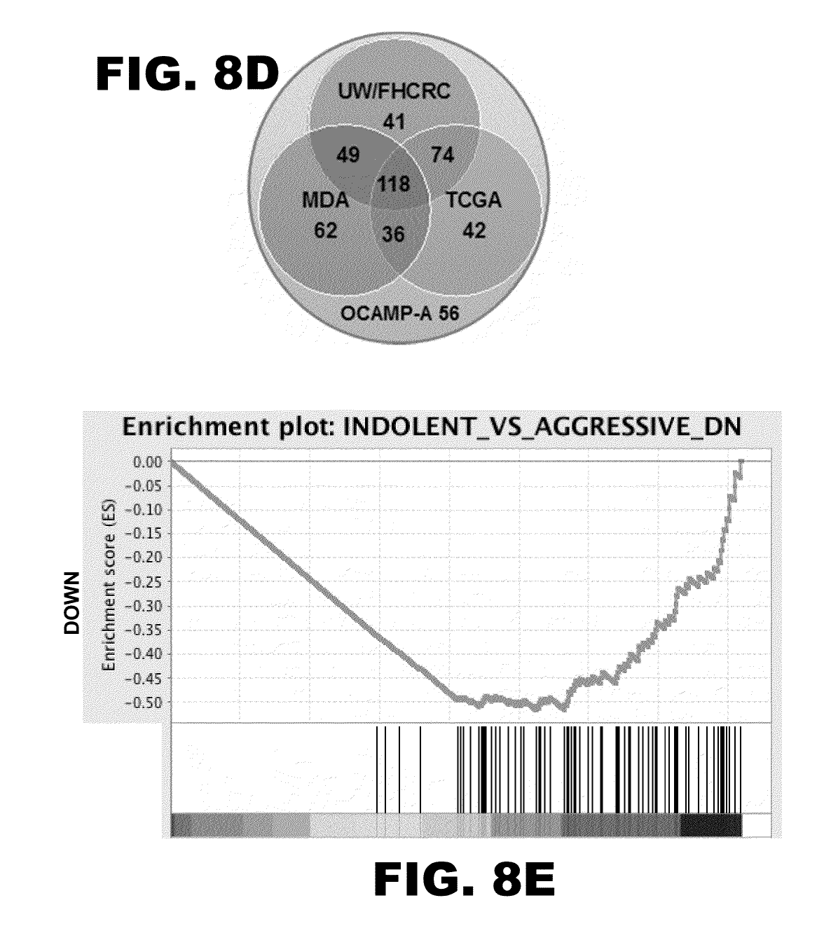

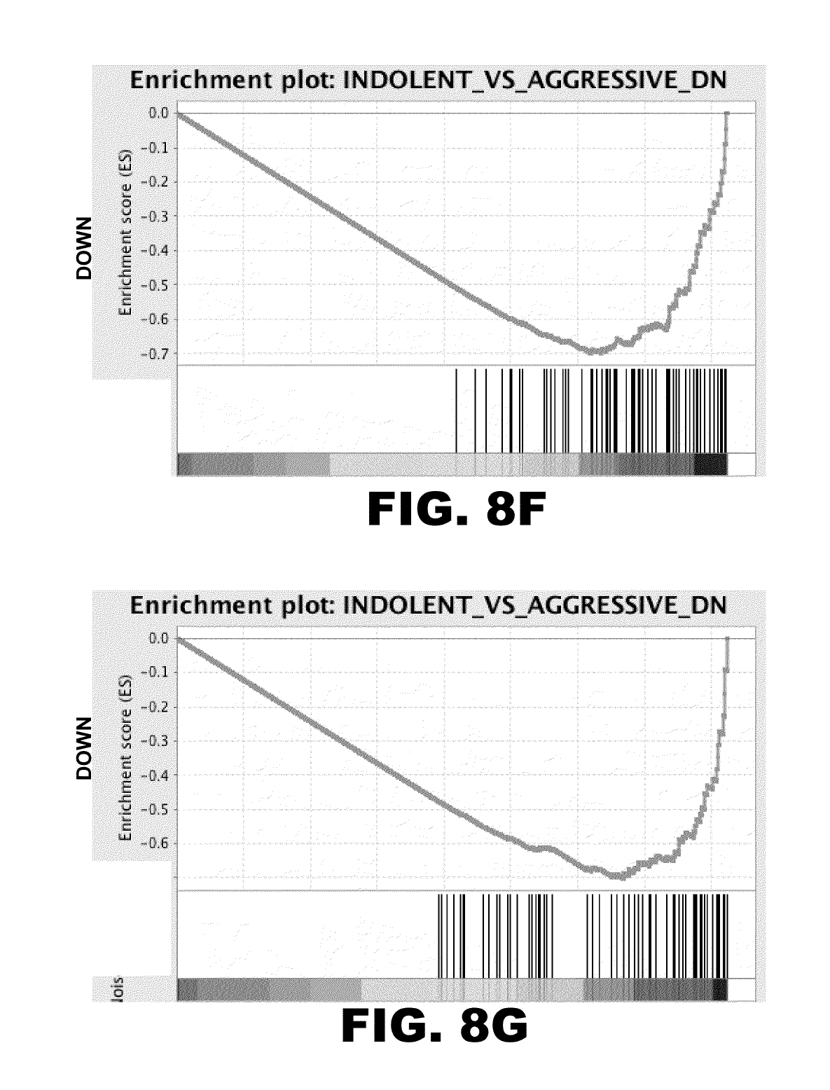

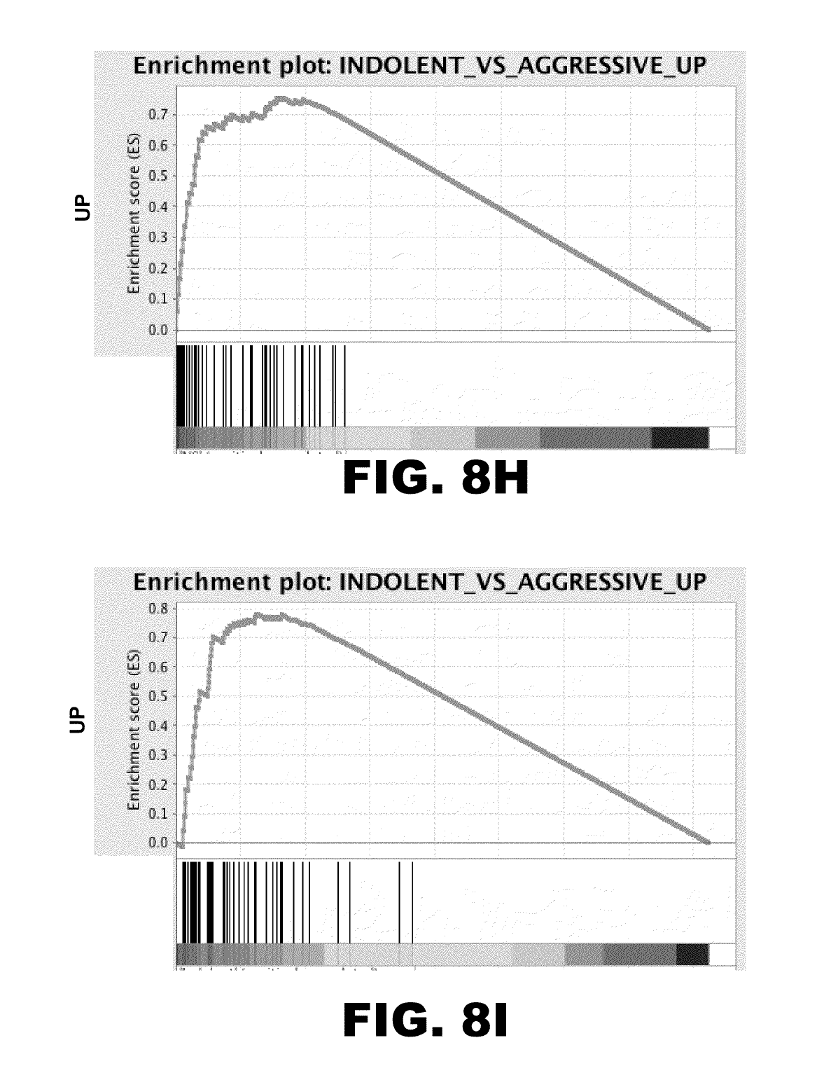

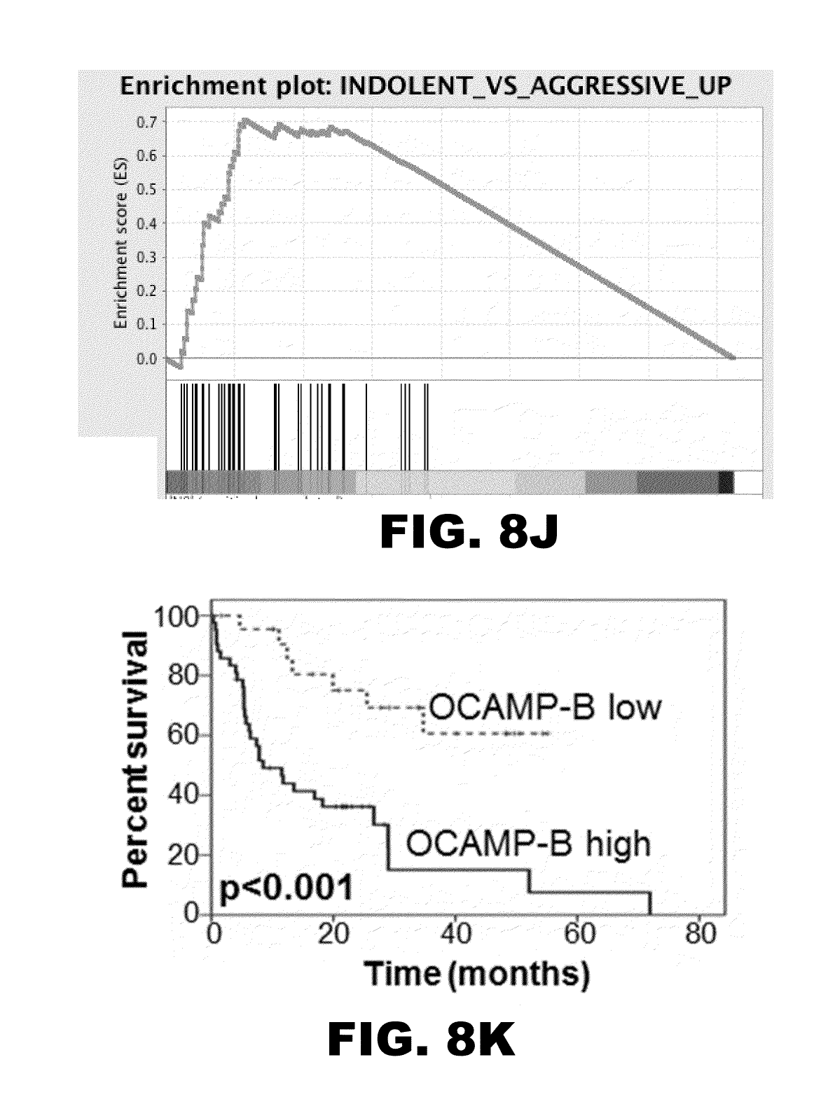

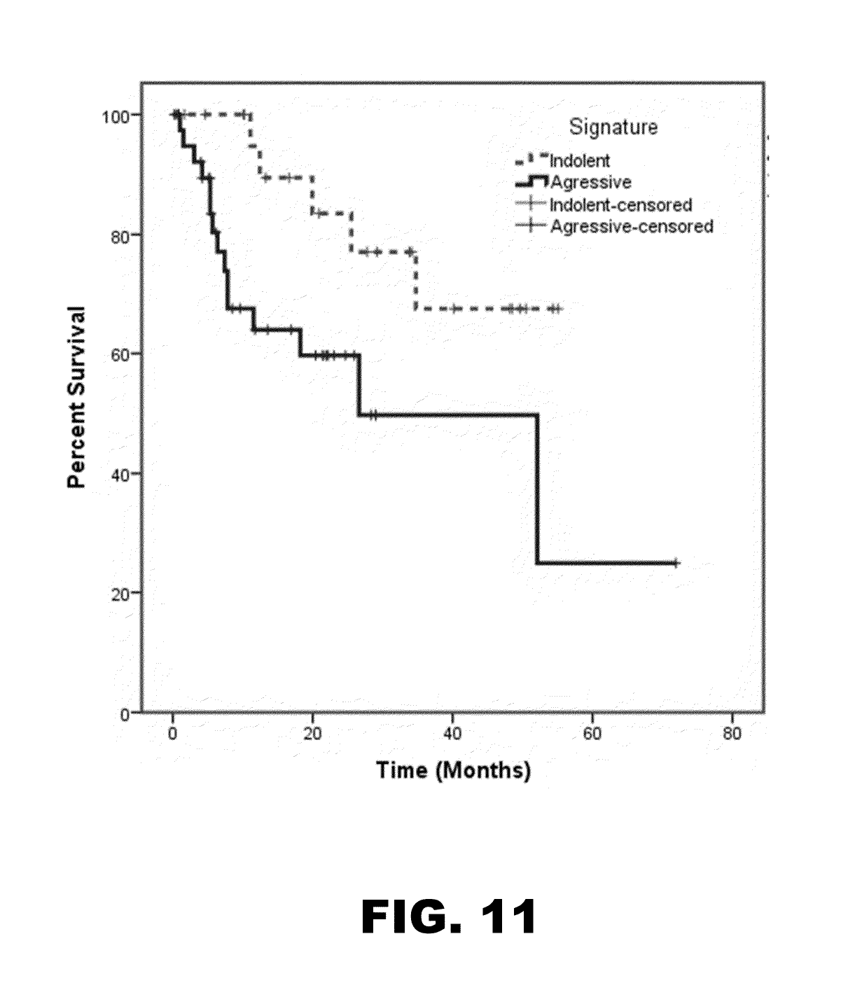

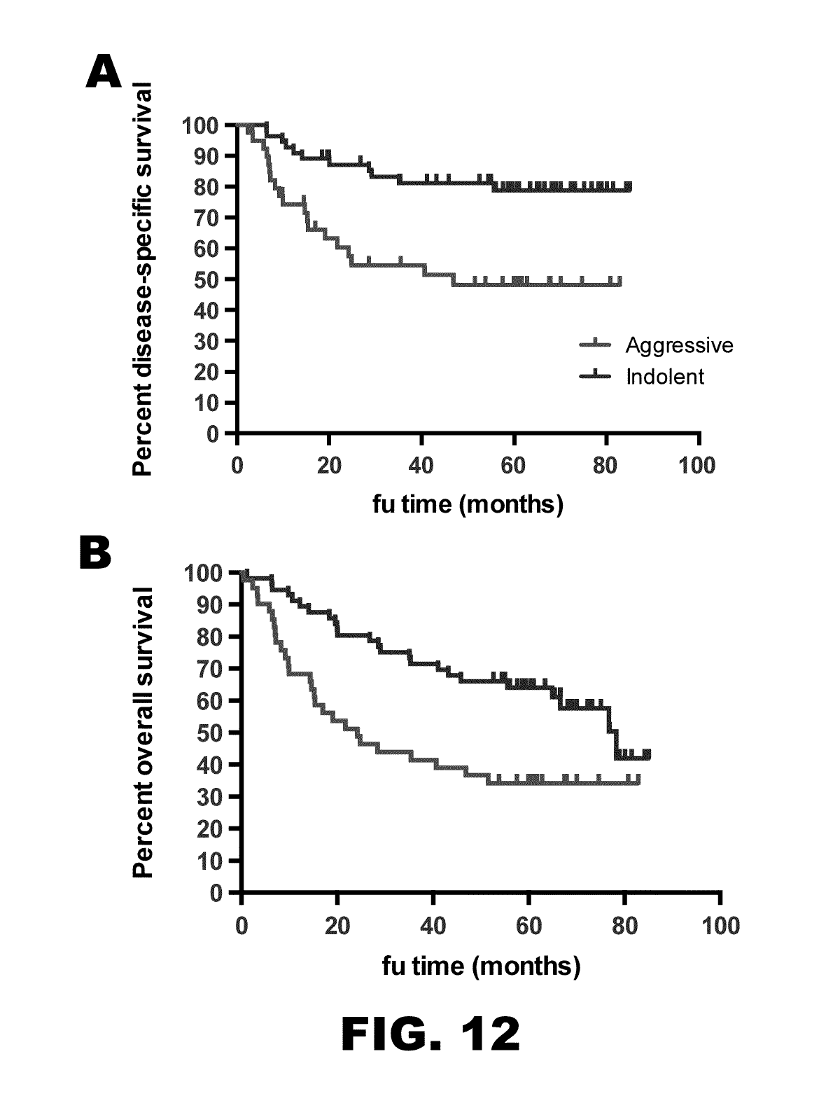

Despite the high p-value for the MD dataset, the inventors noted substantial overall of enriched genes among the three human datasets. The inventors used iterative GSEA based enrichment (FIG. 8A-C, FIG. 7A-H) to identify commonly enriched genes in all three human datasets and eliminate mouse specific transcripts (FIG. 8D,E-J) designated OCAMP-B (118 genes). Because initial analysis for the MD set were not significant, the inventors reassessed significance of OCAMP-B. Kaplan-Meier analysis showed a statistically significant worse overall and disease specific survival for patients with the aggressive OCAMP-B signature (FIG. 8K, (p<0.001), FIG. 11 (p<0.05)). Similar analysis on OSCC patients from the TCGA dataset was limited by the availability of follow-up data. However, OCAMP-B was predictive of both OS and DSS in the UW/FHCRC dataset (p<0.01, FIG. 12A,B).

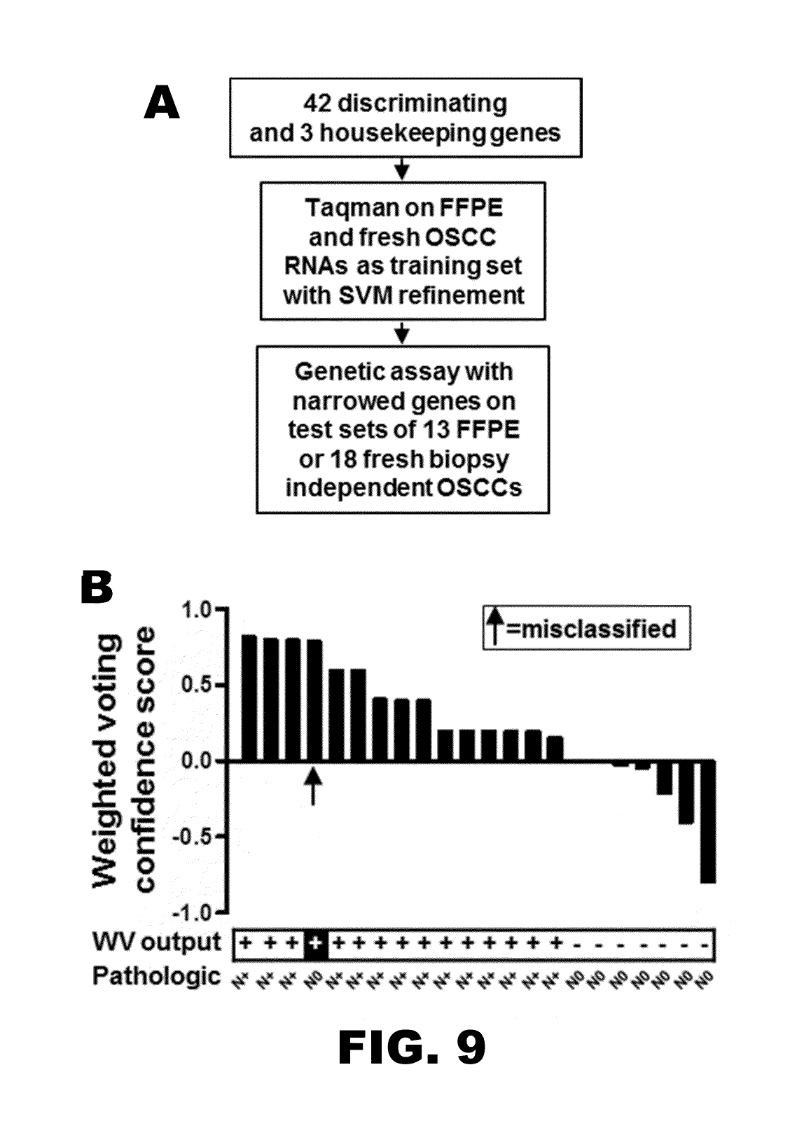

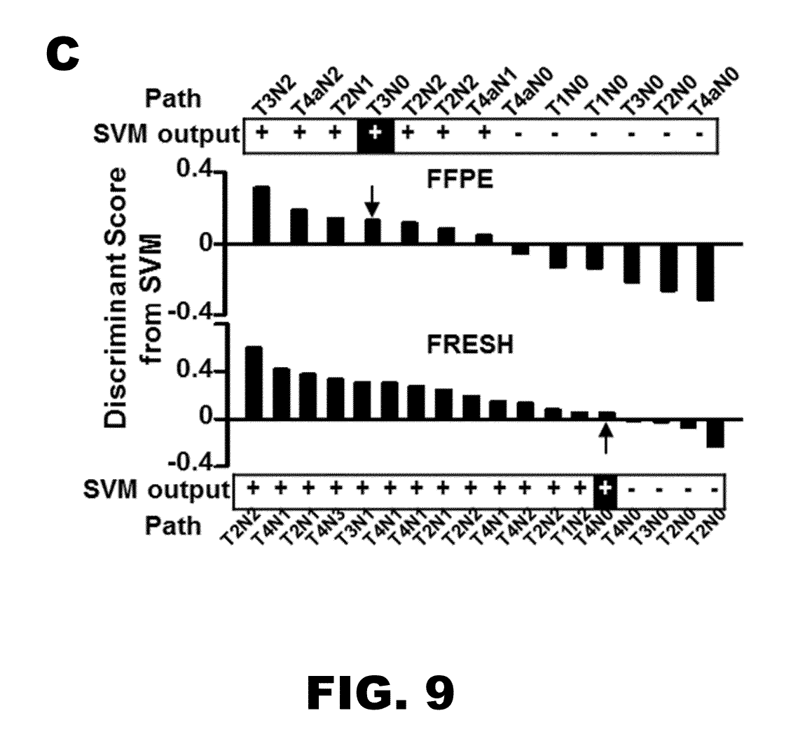

In current OSCC management, clinically node negative patients (the cN0 patient—i.e. those with no suspicious neck lymph nodes by palpation or imaging) undergo neck dissection surgery depending on specific features of the primary tumor to pathologically identify occult nodal metastases. As this approach leads to unnecessary surgery in nearly 80% of patients, the goal of the inventors' is to identify gene expression in the primary tumor predictive of outcomes and occult metastatic disease among newly-diagnosed and untreated patients. Thus, the inventors used clinical rather than pathological TNM staging (available only for the MD Anderson dataset) and found that OCAMP-B status defines unique prognostic subgroups within clinical stages 1/2 and 3/4 (FIG. 10A and Table 18). Multivariate modeling showed a statistically significant independent effect of OCAMP-B such that patients with an aggressive signature were 3.9 times more likely to die (adjusted for TNM hazard ratio value, 95% CI (1.52 to 10.03), Table 19). Finally, the inventors sought to compare the performance of the OCAMP-B signature to histopathological grading. Of 18 patients who were cNO but pathologically N₊ (pN₊, i.e., clinically did not have nodal disease but harbored disease on pathologic analysis after neck dissection), all 18 had the aggressive gene signature. Additionally, of 24 cN₊ and pN₊ patients, all harbored the OCAMP-B aggressive signature. Finally, of 24 patients who were cNO and pN0, all had the indolent signature (FIG. 10B, Table 20). Given that OCAMP-B was generated with overlaps between 3 datasets, the above stratification was not surprising. Towards independent confirmation of OCAMP-B performance, the inventors used a 22 patient OSCC dataset from UPENN³⁰ and saw excellent stratification (21/22 tumors correct) with respect to lymph node metastatic status (FIG. 9B, Table 19). Robust follow-up data for more complex analysis were not reported for this dataset. Together, these findings demonstrate that OCAMP-B allows disease outcome stratification at initial presentation based on results from the primary tumor.

### Example 8. A Multi-Nucleic Acid Assay to Stratify OSCC by Lymph Node Status

As knowledge of lymph node metastatic status of OSCC is critical in clinical decision making, including whether to suggest neck surgery for early stage cancers, the inventors next asked whether the mouse signature could be translated into a diagnostic test as described for ocular melanoma (FIG. 9B,¹⁷). For training sets, the inventors used 17 formalin fixed, paraffin embedded (FFPE) or 16 fresh biopsies specimens from the primary tumor of Washington University OSCC patients with known pathologic status. Using a Taqman platform and a support vector machine-learning algorithm (SVM³¹), 42 discriminating genes were refined into 19 or 10 that classified the FFPE or fresh tumor set with 100% accuracy, respectively. Test sets of 13 independent FFPE or 18 fresh biopsy tumors were then subjected to the assay and analyzed as unknowns by the trained SVM. Accurate lymph node classification of 12/13 FFPE or 17/18 fresh tumors was achieved (FIG. 9A, Table 21, Table 22). Importantly, no N₊ samples were classified as NO and the two NO samples classified as N₊ were from larger T3 and T4 tumors. These data represent proof-of-principle that the OCAMP can be translated for clinical stratification of OSCC patients.

### Discussion for Examples 1-8

Here, the inventors used genomic approaches including exome sequencing and transcriptional profiling to delineate the genetic basis of aggressive growth in the MOC model and in particular focused on its fidelity with human OSCC. Two obvious constraints of their approach were the limited number of different lines and that the aggressive lines were related. Despite these limitations, the MOC lines manifested the breadth of clinical scenarios observed in human OSCC. Their data showed that MOC lines as a group contained mutations in the majority of commonly mutated HNSCC genes, in driver pathways described in human OSCC and in addition, highlighted potential new driver mutations. However, no recurrent mutations associated with aggressive growth were identified. However, transcriptomic analysis revealed a mouse metastasis signature that contained both known and novel candidates for promoters of aggressiveness. Even though this signature was derived from a small number of cell lines, the inventors were surprised it was conserved in three independent human datasets including from the ongoing TCGA effort. Using iterative GSEA, the inventors then developed a consensus 118-transcript metastasis predictor. Finally, using the mouse signature, the inventors were able to develop a preliminary clinically applicable test for genetic stratification of OSCC. Thus, these data have significant potential implications for understanding the biology, prognosis and therapy of human OSCC.

Recent genomics studies to define distinct HNSCC oncogenic driver classes revealed a major functional role for the PI3K-pathway^{10, 12}.The inventors found P13K-pathway mutations in MOC22 and 23 but their functional relevance has not been evaluated. As expected, all MOC lines shared RAS pathway mutations due to the predilection of DMBA for RAS mutations²⁰. Relevant to the HRAS mutant group of human OSCC¹², MOC22 was found to have both HRAS and CASP8 mutations. Interestingly, KRAS was mutated in aggressive MOC lines and NRAS in MOC23; however, these alleles are less common in human OSCC. Importantly, based on the initial description of enhanced ERK1/2 activation in CD44⁺ aggressive lines²⁰, the inventors have initiated a MEK inhibitor (trametinib) clinical trial in patients with OSCC (NCT01553851). Future studies will address the functional contribution of putative drivers. Their focus on the genetic contribution of a conserved mouse to human transcriptional signature supports the existence of a distinct program of aggressiveness in MOC2 and MOC2-10 and human OSCC that is independent of common driver mutations.

Analysis of the aggressiveness biomarker panel revealed several intriguing candidate promoters, most notably the lineage specific transcription factor Nkx2-3 that, in addition to other tissues, is normally expressed in the developing tongue, floor of mouth and mandible³². The NKX family of homeodomain transcription factors has been implicated in a variety of malignancies with lung adenocarcinoma serving as a prime example where Nkx2-1 has a dual role in tumor promotion and metastasis³³. Interestingly, recent work has shown that the Foxal pioneer factor partners with Nkx2-1³⁴ and their analysis shows that Foxal is also upregulated in Nkx2-3 expressing aggressive tumors. Finally, with regard to MOC2-10, the inventors identified Hoxb7, which has been implicated in poor outcomes in OSCC³⁵ and Bmp4, which promotes breast cancer metastasis³⁶, as candidate regulators of lung metastasis in OSCC. Thus, this approach of murine modeling is highly useful, and it supports the generation of additional lines to assess the frequency of recurrent mutations, to extend genotype-phenotype correlations, and to undertake further detailed mechanistic work. Finally, while carcinogenesis with DMBA results in a high number of mutations, it clearly identifies conserved cross-species pathways in contrast to defined oncogene-driven models, perhaps because it allows the natural biology of OSCC to emerge.

Several groups have used expression analyses on human OSCC specimens, with or without lymph node metastasis, to develop predictive genetic biomarkers^{15,16,19}. Van Hooff et al. prospectively showed that their signature had 86% sensitivity and 44% specificity for metastasis in early stage OSCC¹⁹. This signature had an 89% negative predictive value for metastasis in early stage OSCC lesions, but clinical application of the test would still result in either under or overtreatment of significant numbers of patients. Thus, the exact utility of this assay in clinical practice remains to be defined and more robust assays are desirable. The OCAMP signature offers a unique biomarker for human OSCC, as it does not have significant overlap with work described to date (Table 23). The inventors successfully translated the OCAMP signature into a robust assay using a straightforward platform and anticipate rapid progression to larger samples and eventual validation in a prospective fashion. Further work focused on defining the molecular basis of OSCC aggressiveness using the high-fidelity MOC platform may identify additional novel therapeutic approaches for human OSCC.

### Methods for Examples 1-8

Study Approval—Mouse studies were performed and human specimens were obtained under approved protocols of Washington University Animal Studies and the Human Research Protection Office, respectively.

MOC cell line model—Cell lines were generated, characterized and propagated as described²⁰. Further analysis since their initial description revealed that the MOC7 and MOC10 lines were derived from MOC2 and were thus renamed MOC2-7 and MOC2-10 (data not shown). MOC2LN was generated from a lymph node bearing metastatic MOC2. Primary C57BL/6 oral keratinocytes were generated by microdissection of oral mucosa from wild type mice (Taconic), generating single cell suspensions and growing to near confluence using keratinocyte media (CelINTec). Media was then changed to MOC line media for 24 hours prior to RNA isolation.

Exome Capture and Sequencing—Genomic DNA from MOC cells was extracted using DNeasy Blood & Tissue Kit (Qiagen) and was constructed into Illumina libraries according to the manufacturer's protocol (Illumina Inc, San Diego, Calif.). Illumina libraries were processed for analysis on an Illumina GAllx. One microgram of the size-fractionated Illumina library was hybridized to the Agilent mouse exome reagent. After the 24-hour, 42° C. hybridization, the inventors added DynaBeads Streptavidin-coated magnetic beads to selectively remove the biotinylated Agilent probes and hybridized cDNA library fragments. The beads were washed, and the captured library fragments were released into solution using NaOH. The recovered fragments then were PCR amplified according to the manufacturer's protocol using 11 cycles in the PCR. Illumina library quantification was completed using the KAPA SYBR FAST qPCR Kit (KAPA Biosystems, Woburn, Mass.). The qPCR result was used to determine the quantity of library necessary to produce 180,000 clusters on a single lane of the Illumina GAllx. One lane of 100bp paired-End data was generated for each captured sample on the HiSeq 2000 (Illumina).

Mutation Detection and Annotation—As normal tissue from the mice bearing the parental tumors was not available, these mutation calls were compared to the reference C57BL/6 genome for MOC1, 22 and 23 or to the CXCR3^{-/-} exome that the inventors generated in this analysis for MOC2 and 2-10. Sequence data from each tumor and the C57BL/6 genome were aligned independently to NCBI Build 37 of the mouse reference using BWA 0.5.9 and de-duplicated using Picard 1.29 (http://picard.sourceforge.net). Sample variants were called using Samtools (Version 0.1.7a (revision #599)). Somatic single nucleotide variants were detected using VarScan 2 (varscan.sourceforge.net) with the following parameters: min-coverage 30-min-var-freq 0.08 -normal-purity 1 -p-value 0.10-somatic-p-value 0.001-validation 1) and SomaticSniper. Somatic indels were extracted using GATK (Version 3 genome.cshIp.org/cgi/reprint/gr.107524.110v1) and Pindel. All predicted variants were filtered to remove false positives due to potential homopolymer artifacts (variants found in homopolymers with sequence length ≧5 were removed), strand specific sequence artifacts, ambiguously mapped data (the average mapping quality difference between the reference supporting reads and variant supporting reads is greater than 30), and low quality data at the beginning and end of reads (variants supported exclusively by bases observed in first or last 10% of the reads). Variants with an allele frequency <8% were removed. Initial variant transcript annotation was based NCBI mouse build37. Due to lack of a true matched normal tissue, the inventors had more somatic SNPs than expected, so the inventors removed “clustered” SNPs using their internal cluster filter, which allowed a maximum of 2 variants per 0.5 MB genome region and also filtered out mouse dbSNPs. To identify any sample specific mutations, variant allele frequency was calculated for all the SNVs using an internally developed tool Bam2ReadCount (unpublished), which counts the number of reads supporting the reference and variant alleles. The inventors accessed TCGA HNSCC mutational data from (gdac.broadinstitute.org/runs/analyses latest/reports/cancer/HNSC-TP/MutSigNozzleReportCV/nozzle.html).

Microarray—MOC line and primary oral keratinocyte total RNA was isolated using the RNeasy kit (Qiagen) and subjected to gene expression profiling using Illumina MouseRef-8 Expression BeadChips (Illumina, San Diego, Calif.). Raw expression data were subjected to cubic spline normalization in GenomeStudio (version 2011.1). Microarray data are available in NCBIs GEO (GSE50041). Principal component analysis (PCA), ANOVA and hierarchical clustering were performed with Partek Genomics Suite (version 6.6) using a significance of p<0.01 as a threshold for gene inclusion. Significance Analysis of Microarrays (SAM), Version 4.0 was used to generate a ranked gene list, and a threshold of q<10% was then used to select the most highly significant genes that were up or down regulated in indolent versus aggressive mouse cell lines. These lists were used as signature gene sets for Gene Set Enrichment Analysis (GSEA). Human OSCC expression datasets were accessed via public databases and information regarding patient selection, demographics, tumor staging and treatment outcomes were reported in their original publications or on the TCGA data portal.

qRT-PCR-Total RNA was isolated from MOC cell lines (RNeasy, Qiagen) and converted to cDNA using the High Capacity cDNA Reverse Transcription kit (ABI). Taqman nucleic acid expression assays with GAPDH controls were then performed in duplicate using the Taqman Fast advanced master mix (ABI) on an ABI Step One Plus. Relative expression for each probe was then calculated using the comparative Ct method.

Iterative GSEA-based enrichment—Gene Set Enrichment Analysis software and a complete description of the algorithm are provided online by the Broad Institute (broadinstitute.org/GSEA,²¹). Each published OSCC dataset was formatted for GSEA and classified by regional lymph node involvement or stage. GSEA was applied to each dataset using the two lists of significantly up- and down-regulated genes in indolent versus aggressive mouse cell lines. The enrichment scores assigned by GSEA were then used to trim away genes that were oppositely enriched to produce two new, trimmed ranked gene lists derived from each human dataset. GSEA was performed again using the trimmed lists from each dataset against each of the other human datasets; e.g., the lists trimmed by the FHCRC dataset were tested against the MDA dataset, and vice versa, resulting in six pairs of lists with enrichment in two human datasets. This process was continued for another round, producing the final lists that had been trimmed based on enrichment of the mouse genes in all three human expression sets.

Development of clinical assay and SVM analysis-Five FFPE sections (10 μM) each from surgically treated OSCC patients were obtained and tumor areas marked by a board certified head and neck pathologist (JSL). These areas were microdissected and combined for each individual tumor. RNA was harvested using RecoverAll (Ambion) and converted to cDNA using the High Capacity cDNA Reverse Transcription kit (ABI). Using pooled Taqman nucleic acid expression assays (for 42 discriminating and 3 housekeeping nucleic acids (GAPDH, ACTIN and UBC)) and Taqman Pre-Amp master mix (ABI) all samples were pre-amplified for 14 cycles. Samples were then diluted 20-fold and assayed in duplicate for individual nucleic acids using Taqman probes and Gene expression master mix on an ABI Step One Plus. ACt values were calculated by subtracting the geometric mean of the mean Ct values of the three endogenous control nucleic acids from the mean ACt of each discriminating nucleic acid. The 42 nucleic acids were refined into the 19-nucleic acid set by SVM analysis (http://www.chibi.ubc.ca/cgi-bin/nph-SVMsubmit.cgi) on a 17-tumor training set with known pathologic status. SVM was able to accurately classify the 17 tumors when submitted as unknowns using the 19-nucleic acid set data. With the trained SVM, the inventors then submitted data from 13 independent tumors, again with known pathologic status, as unknowns for classification.

For fresh biopsy samples, the inventors acquired RNA prepared from freshly frozen OSCC tumor samples at surgery from the Siteman Cancer Center Tissue Procurement Core. RNA was processed as above for analysis with discriminating and housekeeping genes. The inventors were able to refine the 42 genes into a 10-gene list for fresh samples. Using these probes, the inventors trained the SVM with new data from 16 fresh biopsy tumors. Subsequently, 18 independent test set OSCCs were analyzed and stratified as above.

Statistics—Weighted voting was performed using GenePattern version 3.3.3 for classification of human tumor microarray data (www.broadinstitute.org/cancer/software/genepattern). For weighted voting, the gene expression data for the OCAMP-A signature genes were collected from the UW/FHCRC published dataset. Weighted voting was performed on the entire set, followed by leave-one-out cross-validation, which identified a subset of 26 tumors with correct calls and high confidence (>0.4). This subset was then used as a training set to re-classify the rest of the samples. After identifying the 118 genes of the OCAMP-B signature, leave-one-out cross-validation was performed using the weighted voting algorithm to re-classify samples according to the OCAMP-B signature. Kaplan-Meier survival analysis was performed on the re-classified UW/FHCRC and MDA samples using clinical follow-up data available with the datasets. Statistical analysis was performed in IBM-SPSS (v20.0). Cross tabulation was used to explore the relationship of OCAMP with clinical TNM stage. The impact of gene signature on survival was evaluated using the product limit Kaplan-Meier method.

### References for Examples 1-8

1. Brandwein-Gensler M, Teixeira M S, Lewis C M, Lee B, Rolnitzky L, Hille J J, et al. Oral squamous cell carcinoma: histologic risk assessment, but not margin status, is strongly predictive of local disease-free and overall survival. Am J Surg Pathol. 2005;29(2):167-78.
2. Ganly I, Goldstein D, Carlson D L, Patel SG, O'Sullivan B, Lee N, et al. Long-term regional control and survival in patients with “low-risk,” early stage oral tongue cancer managed by partial glossectomy and neck dissection without postoperative radiation: the importance of tumor thickness. Cancer. 2013;119(6):1168-76.
3. Kalnins I K, Leonard A G, Sako K, Razack M S, Shedd D P. Correlation between prognosis and degree of lymph node involvement in carcinoma of the oral cavity. Am J Surg. 1977;134(4):450-4.
4. Myers J N, Greenberg J S, Mo V, Roberts D. Extracapsular spread. A significant predictor of treatment failure in patients with squamous cell carcinoma of the tongue. Cancer. 2001;92(12):3030-6.
5. Allen C T, Law J H, Dunn G P, Uppaluri R. Emerging insights into head and neck cancer metastasis. Head Neck. 2012.
6. Monroe M M, Gross N D. Evidence-based practice: management of the clinical node-negative neck in early-stage oral cavity squamous cell carcinoma. Otolaryngol Clin North Am. 2012;45(5)1181-93.
7. Mroz E A, Rocco J W. Gene expression analysis as a tool in early-stage oral cancer management. J Clin Oncol. 2012;30(33):4053-5.
8. Rothenberg S M, Ellisen L W. The molecular pathogenesis of head and neck squamous cell carcinoma. J Clin Invest. 2012;122(6):1951-7.
9. Agrawal N, Frederick M J, Pickering C R, Bettegowda C, Chang K, Li R J, et al. Exome sequencing of head and neck squamous cell carcinoma reveals inactivating mutations in NOTCH1. Science. 2011;333(6046):1154-7.
10. Lui V W, Hedberg M L, Li H, Vangara B S, Pendleton K, Zeng Y, et al. Frequent mutation of the PI3K pathway in head and neck cancer defines predictive biomarkers. Cancer Discov. 2013.
11. Stransky N, Egloff A M, Tward A D, Kostic A D, Cibulskis K, Sivachenko A, et al. The mutational landscape of head and neck squamous cell carcinoma. Science. 2011 ;333(6046):1157-60.
12. Pickering C R, Zhang J, Yoo S Y, Bengtsson L, Moorthy S, Neskey D M, et al. Integrative Genomic Characterization of Oral Squamous Cell Carcinoma Identifies Frequent Somatic Drivers. Cancer Discov. 2013.
13. Morris L G, Kaufman A M, Gong Y, Ramaswami D, Walsh L A, Turcan S, et al. Recurrent somatic mutation of FAT1 in multiple human cancers leads to aberrant Wnt activation. Nat Genet. 2013;45(3):253-61.
14. India Project Team of the International Cancer Genome C. Mutational landscape of gingivo-buccal oral squamous cell carcinoma reveals new recurrently-mutated genes and molecular subgroups. Nature communications. 2013;4:2873.
15. Bhattacharya A, Roy R, Snijders A M, Hamilton G, Paquette J, Tokuyasu T, et al. Two distinct routes to oral cancer differing in genome instability and risk for cervical node metastasis. Clinical cancer research : an official journal of the American Association for Cancer Research. 2011;17(22):7024-34.
16. Lohavanichbutr P, Mendez E, Holsinger F C, Rue T C, Zhang Y, Houck J, et al. A 13-gene signature prognostic of HPV-negative OSCC: discovery and external validation. Clinical cancer research : an official journal of the American Association for Cancer Research. 2013;19(5):1197-203.
17. Onken M D, Worley L A, Tuscan M D, Harbour J W. An accurate, clinically feasible multi-gene expression assay for predicting metastasis in uveal melanoma. J Mol Diagn. 2010;12(4):461-8.
18. Paik S, Shak S, Tang G, Kim C, Baker J, Cronin M, et al. A multigene assay to predict recurrence of tamoxifen-treated, node-negative breast cancer. The New England journal of medicine. 2004;351(27):2817-26.
19. van Hooff S R, Leusink F K, Roepman P, Baatenburg de Jong R J, Speel E J, van den Brekel M W, et al. Validation of a gene expression signature for assessment of lymph node metastasis in oral squamous cell carcinoma. J Clin Oncol. 2012;30(33):4104-10.
20. Judd N P, Winkler A E, Murillo-Sauca O, Brotman J J, Law J H, Lewis J S, Jr., et al. ERK1/2 Regulation of CD44 Modulates Oral Cancer Aggressiveness. Cancer Res. 2012;72(1):365-74.
21. Subramanian A, Tamayo P, Mootha V K, Mukherjee S, Ebert B L, Gillette M A, et al. Gene set enrichment analysis: a knowledge-based approach for interpreting genome-wide expression profiles. Proc Natl Acad Sci U S A. 2005;102(43):15545-50.
22. Matsushita H, Vesely M D, Koboldt D C, Rickert C G, Uppaluri R, Magrini V J, et al. Cancer exome analysis reveals a T-cell-dependent mechanism of cancer immunoediting. Nature. 2012;482(7385):400-4.
23. Dipple A, Pigott M, Moschel R C, Costantino N. Evidence that binding of 7,12-dimethylbenz(a)anthracene to DNA in mouse embryo cell cultures results in extensive substitution of both adenine and guanine residues. Cancer Res. 1983;43(9):4132-5.
24. Cerami E, Gao J, Dogrusoz U, Gross B E, Sumer S O, Aksoy B A, et al. The cBio cancer genomics portal: an open platform for exploring multidimensional cancer genomics data. Cancer Discov. 2012;2(5):401-4.
25. Schramek D, Sendoel A, Segal J P, Beronja S, Heller E, Oristian D, et al. Direct in vivo RNAi screen unveils myosin IIa as a tumor suppressor of squamous cell carcinomas. Science. 2014;343(6168):309-13.
26. Cordes C, Hasler R, Werner C, Gorogh T, Rocken C, Hebebrand L, et al. The level of secretory leukocyte protease inhibitor is decreased in metastatic head and neck squamous cell carcinoma. Int J Oncol. 2011;39(1)185-91.
27. Nitta T, Sugihara K, Tsuyama S, Murata F. Immunohistochemical study of MUC1 mucin in premalignant oral lesions and oral squamous cell carcinoma: association with disease progression, mode of invasion, and lymph node metastasis. Cancer. 2000;88(2):245-54.
28. Wang J, Zhang K, Grabowska D, Li A, Dong Y, Day R, et al. Loss of Trop2 promotes carcinogenesis and features of epithelial to mesenchymal transition in squamous cell carcinoma. Mol Cancer Res. 2011;9(12):1686-95.
29. Lohavanichbutr P, Houck J, Doody D R, Wang P, Mendez E, Futran N, et al. Gene expression in uninvolved oral mucosa of OSCC patients facilitates identification of markers predictive of OSCC outcomes. PLoS One. 2012;7(9):e46575.
30. O'Donnell R K, Kupferman M, Wei S J, Singhal S, Weber R, O'Malley B, et al. Gene expression signature predicts lymphatic metastasis in squamous cell carcinoma of the oral cavity. Oncogene. 2005;24(7):1244-51.
31. Pavlidis P, Wapinski I, Noble W S. Support vector machine classification on the web. Bioinformatics. 2004;20(4):586-7.
32. Biben C, Wang C C, Harvey R P. NK-2 class homeobox genes and pharyngeal/oral patterning: Nkx2-3 is required for salivary gland and tooth morphogenesis. Int J Dev Biol. 2002;46(4):415-22.
33. Yamaguchi T, Hosono Y, Yanagisawa K, Takahashi T. NKX2-1/TTF-1: An Enigmatic Oncogene that Functions as a Double-Edged Sword for Cancer Cell Survival and Progression. Cancer Cell. 2013;23(6):718-23.
34. Watanabe H, Francis J M, Woo M S, Etemad B, Lin W, Fries D F, et al. Integrated cistromic and expression analysis of amplified NKX2-1 in lung adenocarcinoma identifies LMO3 as a functional transcriptional target. Genes Dev. 2013;27(2)1 97-210.
35. De Souza Setubal Destro M F, Bitu C C, Zecchin K G, Graner E, Lopes M A, Kowalski L P, et al. Overexpression of HOXB7 homeobox gene in oral cancer induces cellular proliferation and is associated with poor prognosis. Int J Oncol. 2010;36(1):141-9.
36. Pal A, Huang W, Li X, Toy K A, Nikolovska-Coleska Z, Kleer C G. CCN6 modulates BMP signaling via the Smad-independent TAK1/p38 pathway, acting to suppress metastasis of breast cancer. Cancer Res. 2012;72(18):4818-28.

Lengthy table referenced here
US20150259751A1-20150917-T00001
Please refer to the end of the specification for access instructions.

Lengthy table referenced here
US20150259751A1-20150917-T00002
Please refer to the end of the specification for access instructions.

Lengthy table referenced here
US20150259751A1-20150917-T00003
Please refer to the end of the specification for access instructions.

Lengthy table referenced here
US20150259751A1-20150917-T00004
Please refer to the end of the specification for access instructions.

Lengthy table referenced here
US20150259751A1-20150917-T00005
Please refer to the end of the specification for access instructions.

Lengthy table referenced here
US20150259751A1-20150917-T00006
Please refer to the end of the specification for access instructions.

Lengthy table referenced here
US20150259751A1-20150917-T00007
Please refer to the end of the specification for access instructions.

Lengthy table referenced here
US20150259751A1-20150917-T00008
Please refer to the end of the specification for access instructions.

Lengthy table referenced here
US20150259751A1-20150917-T00009
Please refer to the end of the specification for access instructions.

Lengthy table referenced here
US20150259751A1-20150917-T00010
Please refer to the end of the specification for access instructions.

Lengthy table referenced here
US20150259751A1-20150917-T00011
Please refer to the end of the specification for access instructions.

Lengthy table referenced here
US20150259751A1-20150917-T00012
Please refer to the end of the specification for access instructions.

Lengthy table referenced here
US20150259751A1-20150917-T00013
Please refer to the end of the specification for access instructions.

Lengthy table referenced here
US20150259751A1-20150917-T00014
Please refer to the end of the specification for access instructions.

Lengthy table referenced here
US20150259751A1-20150917-T00015
Please refer to the end of the specification for access instructions.

Lengthy table referenced here
US20150259751A1-20150917-T00016
Please refer to the end of the specification for access instructions.

Lengthy table referenced here
US20150259751A1-20150917-T00017
Please refer to the end of the specification for access instructions.

Lengthy table referenced here
US20150259751A1-20150917-T00018
Please refer to the end of the specification for access instructions.

Lengthy table referenced here
US20150259751A1-20150917-T00019
Please refer to the end of the specification for access instructions.

Lengthy table referenced here
US20150259751A1-20150917-T00020
Please refer to the end of the specification for access instructions.

Lengthy table referenced here
US20150259751A1-20150917-T00021
Please refer to the end of the specification for access instructions.

Lengthy table referenced here
US20150259751A1-20150917-T00022
Please refer to the end of the specification for access instructions.

Lengthy table referenced here
US20150259751A1-20150917-T00023
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20150259751A1). An electronic copy of the table will also be available from the USPTO upon request and payment of the fee set forth in 37 CFR 1.19(b)(3).

What is claimed is:

1. A method for determining the aggressiveness of head and neck squamous cell carcinoma (HNSCC) in a subject, the method comprising:
(a) providing a test sample from a subject known to have HNSCC;
(b) determining the nucleic acid expression levels in the test sample of at least 10-nucleic acids selected from Table A:
TABLE A

| | | | |
|---|---|---|---|
| BEX2 | TRIM39 | E2F4 | PKIA |
| DSG3 | IL18RAP | PIP5K1A | RGL2 |
| HOXB7 | P4HA2 | DCBLD2 | CYR61 |
| IGF2BP1 | RAB38 | CASP1 | VDR |
| MUC1 | GSTO2 | SYTL4 | STXBP1 |
| EOMES | SART1 | TACSTD2 | P2RY1 |
| NKX2-3 | INSM1 | PDGFA | OLFML2B |
| MEIS1 | STARD13 | TIMP2 | PPFIBP2 |
| UNC13B | MLLT11 | CAPN5 | TIAM1 |
| TDRKH | DISP1 | SIRT5 | AP1M1 |
| FNTA | LYNX1 | TRAFD1 | STARD5 |
| FOXA1 | RAB40C | COLGALT1 | SLC6A9 |
| DMKN | SYTL1 | ME2 | MTMR9 |
| GSTA4 | PGPEP1 | PLA2G15 | EPHX1 |
| IVL | FMNL1 | BBS4 | AQP3 |
| ANKRD1 | ADPRHL2 | RAB3D | PI4KA |
| GSPT2 | CACNB2 | TAF13 | WNT4 |
| KLF2 | MAGEE1 | FARP1 | DHX38 |
| LPAR1 | CDH2 | LASP1 | ASS1 |
| GPX2 | CELF2 | PCOLCE2 | SLPI |
| MGRN1 | CRK | EPHA1 | IMPA2 |
| PCYT1B | HR | FXYD3 | TNNC1 |
| WRB | FAHD2A | ECM1 | CBR3 |

(c) comparing the expression levels of each nucleic acid in (b) to the corresponding reference expression levels of such nucleic acids, wherein differentially expressed levels in the test sample compared to the reference expression levels indicates aggressive HNSCC.

2. The method of claim 1, wherein the at least 10 nucleic acids comprise: BEX2, DSG3, HOXB7, IGF2BP1, MUC1, EOMES, NKX2-3, MEIS1, UNC13B, TDRKH, FNTA, FOXA1, DMKN, GSTA4, IVL, ANKRD1, GSPT2, KLF2, and LPAR1.

3. The method of claim 1, wherein the nucleic acid expression level of the nucleic acids listed in Table A is determined.

4. The method of claim 1, further comprising determining the nucleic acid expression level of one or more nucleic acids used as a normalization control.

5. The method of claim 4, wherein the one or more nucleic acids used as a normalization control are selected from the group consisting of UBC, GAPDH and actin.

6. The method of claim 1, wherein the reference expression level is from a control subject or group of subjects in which HNSCC is known to be indolent.

7. The method of claim 1, wherein the HNSCC is OSCC.

8. The method of claim 7, wherein the test sample is OSCC tumor tissue from a plurality of anatomical sites in the oral cavity of the subject comprising buccal, floor of the mouth (FOM), tongue, alveolar, retromolar, palate, gingival, or other oral tissue.

9. The method of claim 1, wherein the at least 10-nucleic acids further comprises one or more additional nucleic acids selected from Table B:
TABLE B

| | | | | | |
|---|---|---|---|---|---|
| PCOLCE | MKRN3 | GIT1 | STK32C | PHLDA3 | COL12A1 |
| PACSIN2 | LSM11 | SOCS7 | SLC35F2 | PCBP3 | ECE1 |
| CTGF | BCAR1 | IGSF11 | SETD6 | HES1 | THBS2 |
| FGF13 | GCA | TRMT2A | RRAGB | CEBPB | COL16A1 |
| SP8 | IQGAP3 | CAMKK1 | DST | TEAD3 | MET |
| TNPO2 | MSH6 | GSG1 | PTPN21 | SLC1A3 | TNFRSF1A |
| NEK8 | UCK2 | CHN2 | LRP11 | XDH | CLSTN1 |
| RAD23A | EXOC8 | FLII | NISCH | CELSR2 | SOSTDC1 |
| ELL | HAVCR2 | TPD52L1 | CDC42BPB | SNCG | ART4 |
| XPO4 | PDP1 | ASL | PLCB3 | BCAP31 | FZD7 |
| ZFPM2 | FOSL1 | LTK | DNMBP | CD9 | OCIAD2 |
| AKAP12 | DEPDC1B | CCDC109A | SPIRE2 | ATP1A1 | HS6ST1 |
| RPP21 | B3GNT5 | CAMK2B | NADSYN1 | TEF | FST |
| PRMT5 | TPH1 | DDX49 | UNC13D | MMP2 | APP |
| USP13 | ESPL1 | DNAJA2 | DHX32 | MCAT | CLOCK |
| PLCL2 | UPF3A | SYN1 | PRKCZ | COL18A1 | PPP1R14C |
| RBPMS | GMIP | CLCF1 | PLEKHA2 | RHOJ | DLL1 |
| PKN1 | USP43 | PHACTR4 | GM2A | RBMS3 | KIF13A |
| ZFP57 | HIST1H2AE | NOB1 | CHCHD7 | CASQ2 | FJX1 |
| DDAH1 | PCSK9 | LMTK2 | TBX3 | IFT140 | PORCN |
| RIMS2 | WDR6 | PPAP2A | TMEM108 | STAB1 | LAPTM4A |
| UCHL1 | CDC23 | MIA2 | AK1 | LRP1 | KCMF1 |
| ARHGEF18 | DONSON | GFOD1 | OSGEP | AMPD1 | ODZ4 |
| NEO1 | HHEX | NSF | RENBP | SLC39A4 | JAG2 |
| COG1 | TMEM20 | GPS2 | SERPINF1 | RAB15 | SQLE |
| RAB3B | VTI1A | MYO1C | NPHP4 | IFITM2 | ITGB4 |
| KCNF1 | FUT10 | NUDCD3 | UGT1A10 | LSP1 | SLC7A8 |
| DUSP3 | PTTG1 | TACC1 | PTN | PPBP | PPP5C |
| CRYL1 | MARVELD3 | PPA2 | PLD3 | ORM1 | MYO1B |
| CKB | HLCS | NT5C2 | RFX2 | PTMS | EFS |
| CGNL1 | FKBP5 | LRSAM1 | RGMA | APOBEC1 | TAPBP |
| FGD3 | DCAF5 | TRAPPC5 | ADA | SLC5A8 | BNC1 |
| EGLN1 | PPM1D | PRDX2 | ARHGEF19 | DACT2 | BCL7A |
| GPRC5A | PLSCR2 | TJP1 | PBX1 | GSTO1 | INHBB |
| RPS6KL1 | IGF2BP2 | PRODH | TMEM53 | POLR2J | SSFA2 |
| TMEM160 | ARHGAP8 | MTCH2 | NXN | DCXR | IMPACT |
| PDSS1 | MOV10 | ZFPM1 | SCMH1 | VAMP5 | NRTN |
| RIN3 | TDRD7 | EPN2 | NKD2 | PSTPIP1 | FSCN1 |
| RBPMS2 | PBX4 | HNF1B | ROR2 | CYP2S1 | BCL6 |
| GAN | SFXN1 | SCAMP5 | PTPRU | PIGF | ING4 |
| ZDHHC3 | ISG20 | IKBKB | PLXNA2 | CTH | SCP2 |
| GIPC2 | POU4F1 | GALK1 | KLF4 | TSPO | PER2 |
| OXSR1 | KCTD9 | ADK | IFITM3 | NDUFA4 | SLC11A2 |
| TUBB2B | CLCA4 | THYN1 | PPP1CB | MOXD1 | KCTD15 |
| LEPRE1 | FAIM | B3GNT3 | COL23A1 | WTIP | GALNTL4 |
| GLT25D1 | NUP210 | VGF | GYLTL1B | ARF5 | ATP10D |
| NUP133 | TJP3 | MSRB2 | MST1R | RNASET2 | SSBP2 |
| EPS8 | PKP2 | ATP6V0A1 | SULF1 | PRICKLE3 | LIMK2 |
| GJC1 | FGF5 | FGF22 | ATG5 | CCND2 | SOX15 |
| SF3B2 | PEA15 | HCN2 | EXT1 | GPR108 | IRX5 |
| GALNT10 | COL4A2 | SERPIND1 | DAPK1 | RSPH1 | SP6 |
| CHERP | DCLK2 | ABHD14B | PPFIBP1 | ATOX1 | PARD6G |
| CSTF3 | FHOD1 | SAMD10 | CDC42EP3 | SEMA4A | TAPBPL |
| GSTCD | SLC44A4 | BSPRY | VGLL4 | MGST2 | PVRL1 |
| RAB21 | SMG5 | WDR33 | M6PR | HYAL1 | CXCL14 |
| HSD17B7 | CHRNB1 | ANKRD50 | ICAM1 | FXYD4 | GNA15 |
| DUSP11 | CLDN6 | DPP3 | FBXO32 | B3GALT4 | TMEM54 |
| FAS | PRDX4 | F2R | INPP5F | IL17RE | VSNL1 |
| PSMB9 | PPP1R9A | COL4A1 | TNFAIP8 | PLCH2 | RAPGEFL1 |
| MYH10 | LAMB2 | ZCCHC14 | SIK1 | GSTK1 | PGAP2 |
| AQP8 | SPRY3 | SCAMP3 | ST5 | IL17RC | CRABP2 |
| UNC5B | BACE1 | NANOS1 | SORL1 | RARG | TRIM29 |
| RASL11A | PVR | MED10 | OSMR | CSTB | ADORA2B |
| LRP5 | APBB1 | BTBD12 | LY6E | RBAK | GPR64 |
| F2RL1 | ULK1 | CDH6 | SEC61B | IRAK2 | RNF19A |

10. The method of claim 1, wherein determining the expression level comprises use of quantitative PCR, such as quantitative RT-PCR or microarray.

11. The method of claim 1, further comprising treatment of the subject with adjuvant chemotherapy if aggressive HNSCC is indicated.

12. A method of treating a subject in need thereof comprising:
(a) obtaining a test sample from the subject;
(b) determining the aggressiveness of head and neck squamous cell carcinoma (HNSCC) in the subject according to the method of claim 1; and
(c) administering to the subject predicted to have aggressive HNSCC a treatment suitable for aggressive HNSCC.

13. The method of claim 12, wherein the treatment suitable for aggressive HNSCC is adjuvant chemotherapy.

14. The method of claim 12, wherein the HNSCC is OSCC>

15. A kit for determining the aggressiveness of head and neck squamous cell carcinoma (HNSCC) in a subject, the kit comprising:
(a) a substrate for holding a test sample isolated from the subject;
(b) an at least 10-nucleic acid molecular signature selected from Table A:
TABLE A

| | | | |
|---|---|---|---|
| BEX2 | TRIM39 | E2F4 | PKIA |
| DSG3 | IL18RAP | PIP5K1A | RGL2 |
| HOXB7 | P4HA2 | DCBLD2 | CYR61 |
| IGF2BP1 | RAB38 | CASP1 | VDR |
| MUC1 | GSTO2 | SYTL4 | STXBP1 |
| EOMES | SART1 | TACSTD2 | P2RY1 |
| NKX2-3 | INSM1 | PDGFA | OLFML2B |
| MEIS1 | STARD13 | TIMP2 | PPFIBP2 |
| UNC13B | MLLT11 | CAPN5 | TIAM1 |
| TDRKH | DISP1 | SIRT5 | AP1M1 |
| FNTA | LYNX1 | TRAFD1 | STARD5 |
| FOXA1 | RAB40C | COLGALT1 | SLC6A9 |
| DMKN | SYTL1 | ME2 | MTMR9 |
| GSTA4 | PGPEP1 | PLA2G15 | EPHX1 |
| IVL | FMNL1 | BBS4 | AQP3 |
| ANKRD1 | ADPRHL2 | RAB3D | PI4KA |
| GSPT2 | CACNB2 | TAF13 | WNT4 |
| KLF2 | MAGEE1 | FARP1 | DHX38 |
| LPAR1 | CDH2 | LASP1 | ASS1 |
| GPX2 | CELF2 | PCOLCE2 | SLPI |
| MGRN1 | CRK | EPHA1 | IMPA2 |
| PCYT1B | HR | FXYD3 | TNNC1 |
| WRB | FAHD2A | ECM1 | CBR3 |

(c) agents for detection/measurement of the at least 10-nucleic acid molecular signature; and optionally
(d) printed instructions for reacting the agents with the biological sample or a portion of the biological sample to detect the presence or amount of each nucleic acid of the at least 10-nucleic acid molecular signature in the biological sample.

16. The method of claim 15, wherein the at least 10-nucleic acid molecular signature comprises: BEX2, DSG3, HOXB7, IGF2BP1, MUC1, EOMES, NKX2-3, MEIS1, UNC13B, TDRKH, FNTA, FOXA1, DMKN, GSTA4, IVL, ANKRD1, GSPT2, KLF2, and LPAR1.

17. The method of claim 15, wherein the at least 10 nucleic acid-molecular signature consists of Table A.

18. The method of claim 15, further comprising one or more nucleic acids to be used as a normalization control.

19. The method of claim 15, wherein the HNSCC is OSCC.

20. The method of claim 15, wherein the agents for detection/measurement determine the nucleic acid expression level of the 10-nucleic acid molecular signature using quantitative PCR, such as quantitative RT-PCR or microarray.
